(19) United States
(12) Patent Application Publication
(10) Pub. No.: US 2018/0164535 A1
(43) Pub. Date: Jun. 14, 2018

(54) Methods for Display Updates Based on Wavefront Sensing on an Eye

(71) Applicant: Ovitz Corporation, West Henrietta, NY (US)

(72) Inventor: Nicolas Scott Brown, Rochester, NY (US)

(21) Appl. No.: 15/831,327

(22) Filed: Dec. 4, 2017

Related U.S. Application Data
(60) Provisional application No. 62/434,351, filed on Dec. 14, 2016.

(51) Int. Cl. G02B 7/04; A61B 3/10; A61B 3/14; G06K 9/00; G09G 3/00; G02B 27/10

(52) U.S. Cl. CPC G02B 7/04; A61B 3/1015; A61B 3/14; G09G 2354/00; G09G 3/001; G02B 27/10; G06K 9/00604

(57) ABSTRACT

An electronic device includes one or more processors; and memory storing one or more programs. The device receives an image of a light pattern. The light pattern is based on light reflected from a retina of an eye and includes an array of spots. The device determines a refraction value of the eye by analyzing locations of the array of spots in the image, and adjusts the display device based on at least the refraction value of the eye.

### RELATED APPLICATIONS

This application claims priority to, and benefit of, U.S. Provisional Application Ser. No. 62/434,351, filed Dec. 14, 2016, entitled “Methods for Display Updates Based on Wavefront Sensing on an Eye.” This application is related to U.S. patent application Ser. No. 15/247,647, filed Aug. 25, 2016, entitled “Devices and Methods for Wavefront Sensing and Corneal Topography,” which is a continuation application of U.S. patent application Ser. No. 14/928,063, filed Oct. 30, 2015, now U.S. Pat. No. 9,427,150, entitled “Devices and Methods for Wavefront Sensing and Corneal Topography,” which claims priority to, and benefit of, U.S. Provisional Patent Application Ser. No. 62/210,893, filed Aug. 27, 2015, entitled “Devices and Methods for Wavefront Sensing and Corneal Topography.” All of these applications are incorporated by reference herein in their entireties.

### TECHNICAL FIELD

This application relates generally to wavefront sensing, and more particularly, portable devices that are capable of performing wavefront sensing and updating displays.

### BACKGROUND

Eyes are important organs, which play a critical role in human's visual perception. An eye has a roughly spherical shape and includes multiple elements, such as cornea, lens, vitreous humour, and retina. Imperfections in these components can cause reduction or loss of vision. For example, too much or too little optical power in the eye can lead to blurring of the vision (e.g., near-sightedness and far-sightedness), and astigmatism can also cause blurring of the vision.

Wavefront sensors are important tools in ophthalmology. Wavefront sensors provide information indicating one or more aberrations in the eye. In particular, wavefront sensors have an advantage over auto-refractors in that wavefront sensors can measure higher order aberrations.

However, results of wavefront sensing have not been used in updating displays.

### SUMMARY

Accordingly, there is a need for devices that can perform wavefront sensing and update displays based on the results of wavefront sensing. Such devices and related methods optionally complement or replace conventional devices and methods. Such devices provide portability, performance, and convenience that are not available from conventional devices and methods.

The above deficiencies and other problems associated with conventional devices and corresponding methods are reduced or eliminated by the disclosed devices.

As described in more detail below, some embodiments involve an electronic device that includes one or more processors; and memory storing one or more programs for execution by the one or more processors. The one or more programs include instructions for receiving an image of a light pattern. The light pattern is based on light reflected from a retina of an eye and includes an array of spots. The one or more programs also include instructions for determining a refraction value of the eye by analyzing locations of the array of spots in the image; and adjusting the display device based on at least the refraction value of the eye.

In some embodiments, receiving the image of the light pattern includes: transmitting light emitted from a light source toward an eye; transmitting light from the eye through an array of lenses; and receiving, with an image sensor, the light from the eye transmitted through the array of lenses.

In some embodiments, the light emitted from the light source is not transmitted through the array of lenses.

In some embodiments, the device includes the light source, the array of lenses, and the image sensor.

In some embodiments, determining the refraction value of the eye includes comparing the locations of the array of spots in the image with reference locations for the array of spots.

In some embodiments, the display device includes an array of light emission elements and one or more adjustable lenses. Adjusting the display device includes changing a focal length of the one or more adjustable lenses.

In some embodiments, the one or more programs include instructions for, in accordance with a determination that the refraction value of the eye is lower than a reference value, decreasing the focal length of the one or more adjustable lenses.

In some embodiments, the one or more programs include instructions for, in accordance with a determination that the refraction value of the eye is higher than a reference value, increasing the focal length of the one or more adjustable lenses.

In some embodiments, the electronic device is coupled with a pupil camera. The one or more programs include instructions for: obtaining an image of a pupil of the eye with the pupil camera; determining a vergence of the eye from the image of the pupil of the eye; and adjusting the display device based on at least the refraction value of the eye and the vergence of the eye.

In some embodiments, the one or more programs include instructions for: obtaining one or more images of pupils of two eyes with the pupil camera; determining a vergence of the two eyes from the one or more images; and adjusting the display device based on at least the refraction value of the eye and the vergence of the two eyes.

In some embodiments, the device includes the pupil camera.

In accordance with some embodiments, a method is performed at an electronic device with one or more processors, memory, and a display device coupled with the one or more processors. The method includes receiving an image of a light pattern. The light pattern is based on light reflected from a retina of an eye and includes an array of spots. The method also includes determining a refraction value of the eye by analyzing locations of the array of spots in the image; and adjusting the display device based on at least the refraction value of the eye.

In accordance with some embodiments, a computer readable storage medium stores one or more programs for execution by one or more processors of an electronic device. The one or more programs include instructions for receiving an image of a light pattern. The light pattern is based on light reflected from a retina of an eye and includes an array of spots. The one or more programs also include instructions for determining a refraction value of the eye by analyzing locations of the array of spots in the image; and adjusting the display device based on at least the refraction value of the eye.

Thus, electronic devices coupled with display devices are provided with methods for providing sharper images, thereby increasing user satisfaction with such devices. Such devices and corresponding methods may complement or replace conventional methods for updating displays.

### BRIEF DESCRIPTION OF THE DRAWINGS

For a better understanding of the various described embodiments, reference should be made to the Description of Embodiments below, in conjunction with the following drawings in which like reference numerals refer to corresponding parts throughout the figures.

FIG. 1A illustrates optical components of a portable device in accordance with some embodiments.

FIG. 1B illustrates wavefront sensing with the portable device shown in FIG. 1A, in accordance with some embodiments.

FIG. 1C illustrates corneal topography with the portable device shown in FIG. 1A, in accordance with some embodiments.

FIGS. 1D-1G illustrate light sources configured to project an array of spots in accordance with some embodiments.

FIG. 1H illustrates a portable device in accordance with some embodiments.

FIGS. 1I-1J illustrate optical components of a portable device in accordance with some embodiments.

FIG. 1K is a schematic diagram illustrating a portable device including a display device in accordance with some embodiments.

FIG. 2 is a block diagram illustrating electronic components of a portable device in accordance with some embodiments.

FIG. 3 is a block diagram illustrating a distributed computing system in accordance with some embodiments.

FIG. 4 is a flowchart representing a method of optical measurements with a portable device, in accordance with some embodiments.

FIG. 5 is a flowchart representing a method of optical measurements with a portable device, in accordance with some embodiments.

FIG. 6 illustrates exemplary calibration curves for adjusting one or more aberrations of an eye based on a position of the eye relative to the device, in accordance with some embodiments.

FIG. 7 is an exemplary image of an eye with projection of a spot array pattern in accordance with some embodiments.

FIGS. 8A-8D illustrate examples of images collected by a wavefront sensor for eyes having different refraction values.

FIGS. 9A-9E are schematic diagrams illustrating operations of a portable electronic device with a display device in accordance with some embodiments.

FIGS. 10A-10B illustrate a vergence of the eyes in accordance with some embodiments.

FIG. 11 is a flowchart representing a method of determining a refraction value of an eye and adjusting a display device in accordance with some embodiments.

### DESCRIPTION OF EMBODIMENTS

Conventional wavefront sensors are widely used for detecting one or more aberrations of an eye. Conventional corneal topographers are used for determining a profile of a cornea. However, conventional devices that can perform both wavefront sensing and corneal topography have not been made portable. It is not simply the size of the conventional devices that has prevented miniaturization of such devices. Rather, the inventors of this application have observed that the conventional devices, if just reduced in size, would suffer from significant errors. The inventors of this application have discovered that the errors are mainly due to the positioning of the eye relative to the pupil plane of a device. Conventional devices include a bulky mechanism for aligning the position of an eye so that the eye is positioned on the pupil plane. However, such a bulky mechanism cannot be used in portable devices, and without the alignment mechanism, significant errors were observed in miniaturized devices. The inventors of this application have discovered that a new optical design, which includes a lens assembly in a particular position, significantly reduces the impact of the positioning error. Portable devices with such lens assemblies can perform both wavefront sensing and corneal topography with superior performance compared to conventional devices.

Reference will be made to embodiments, examples of which are illustrated in the accompanying drawings. In the following description, numerous specific details are set forth in order to provide a thorough understanding of the various described embodiments. However, it will be apparent to one of ordinary skill in the art that the various described embodiments may be practiced without these particular details. In other instances, methods, procedures, components, circuits, and networks that are well-known to those of ordinary skill in the art are not described in detail so as not to unnecessarily obscure aspects of the embodiments.

It will also be understood that, although the terms first, second, etc. may be used herein to describe various elements, these elements should not be limited by these terms. These terms are only used to distinguish one element from another. For example, a first image sensor could be termed a second image sensor, and, similarly, a second image sensor could be termed a first image sensor, without departing from the scope of the various described embodiments. The first image sensor and the second image sensor are both image sensors, but they are not the same image sensor.

The terminology used in the description of the embodiments herein is for the purpose of describing particular embodiments only and is not intended to be limiting of the invention. As used in the description of the invention and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting (the stated condition or event)” or “in response to detecting (the stated condition or event),” depending on the context.

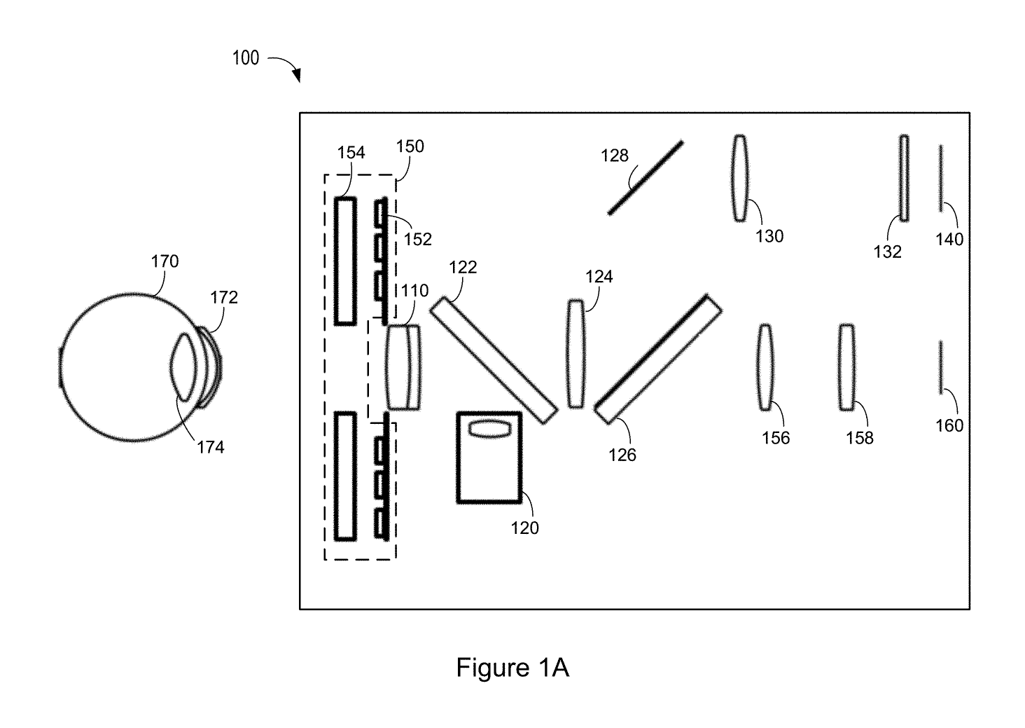

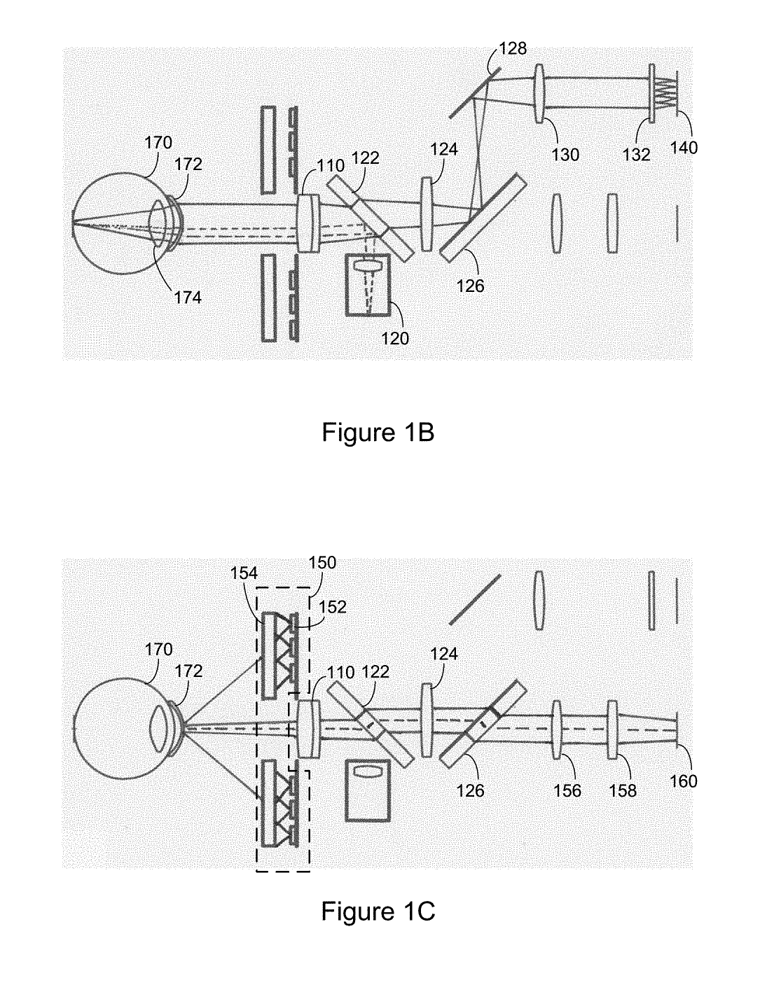

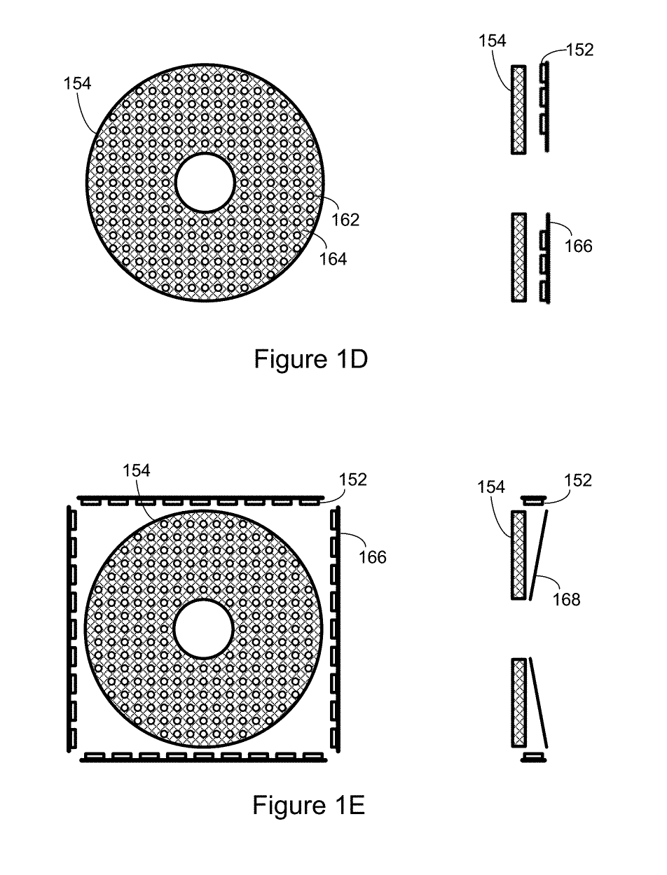

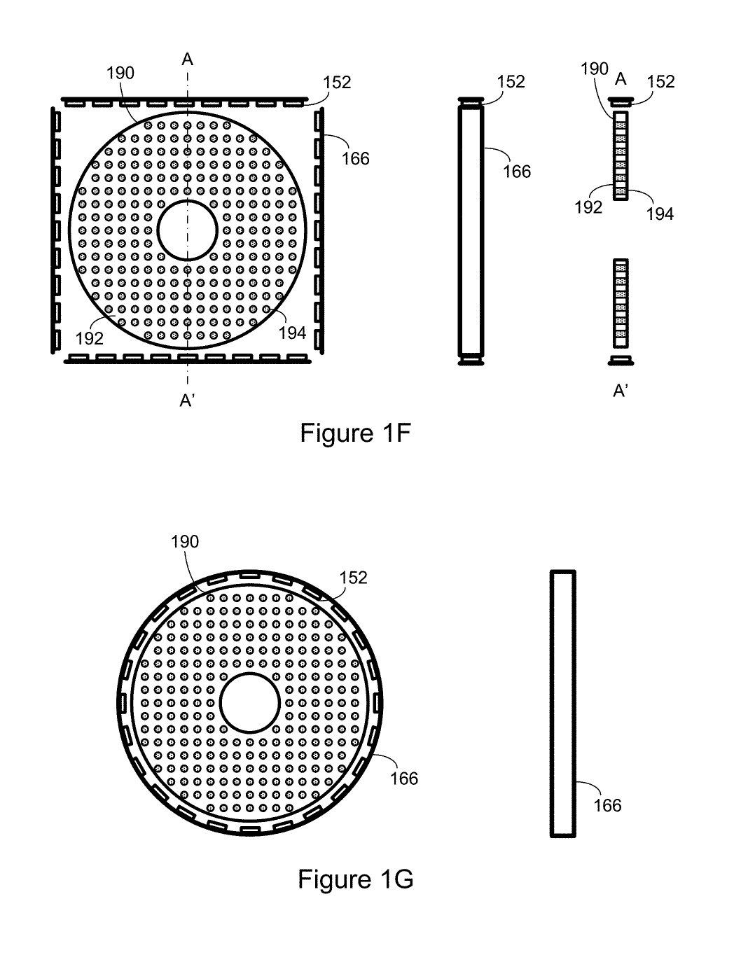

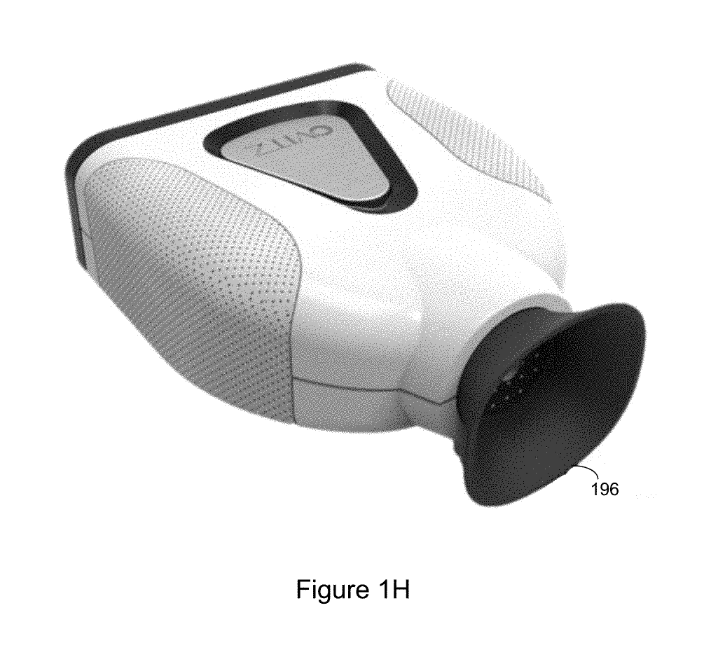

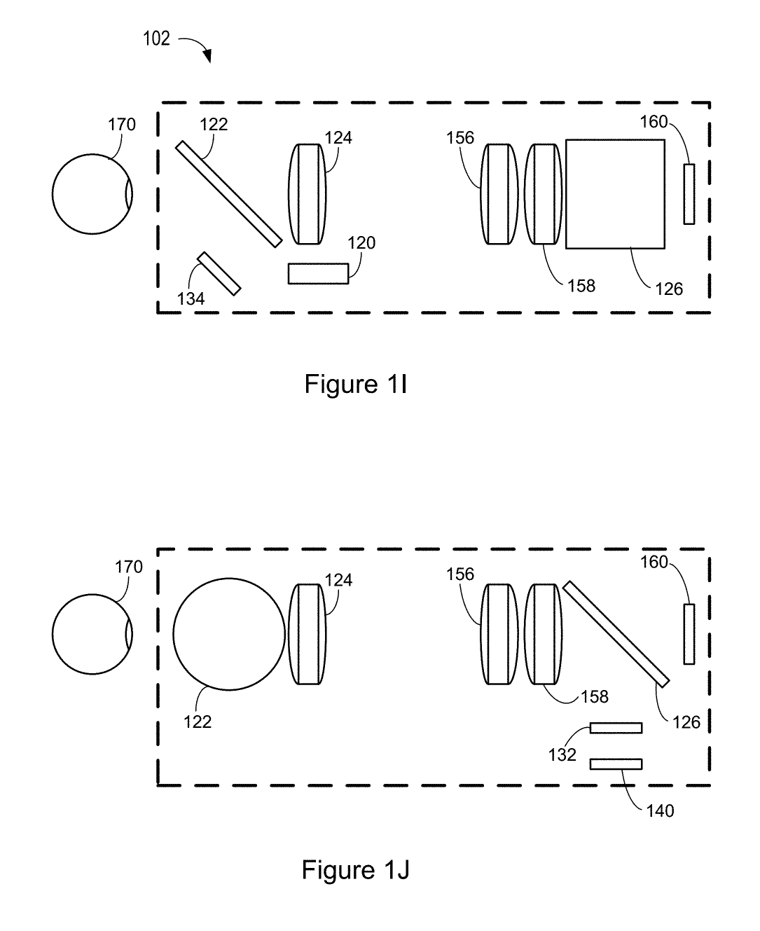

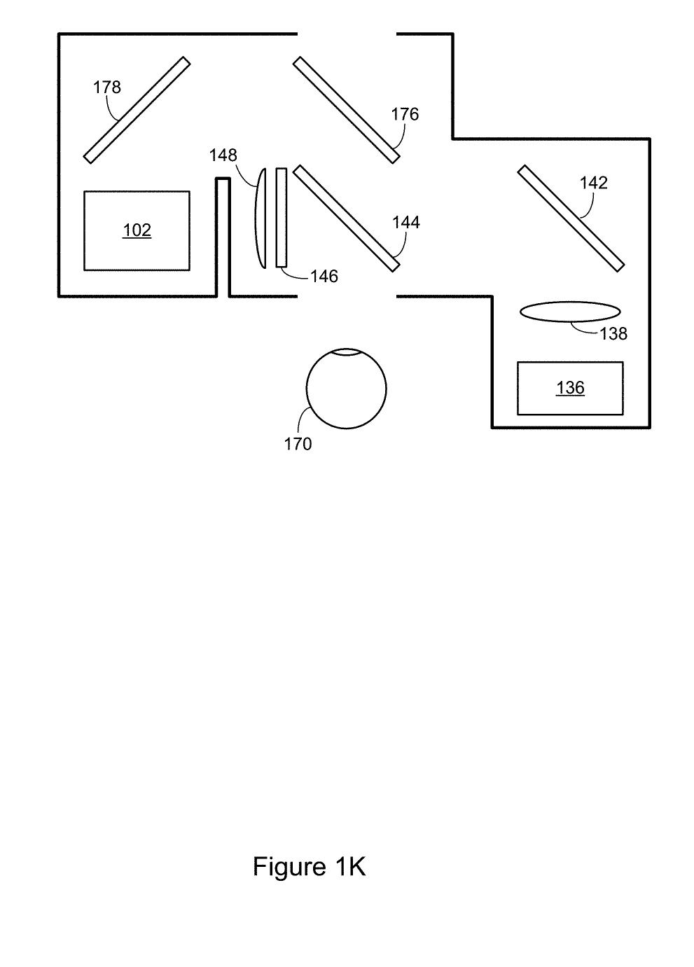

FIG. 1A illustrates optical components of portable device 100 in accordance with some embodiments.

Device 100 includes lens assembly 110. In some embodiments, lens assembly 110 is a doublet lens, as shown in FIG. 1A. For example, a doublet lens is selected to reduce spherical aberration and other aberrations (e.g., coma and/or chromatic aberration). In some embodiments, lens assembly 110 is a triplet lens. In some embodiments, lens assembly 110 is a singlet lens. In some embodiments, lens assembly 110 includes two or more separate lenses. In some embodiments, lens assembly 110 includes an aspheric lens. In some embodiments, a working distance of lens assembly 110 is between 10-100 mm (e.g., between 10-90 mm, 10-80 mm, 10-70 mm, 10-60 mm, 10-50 mm, 15-90 mm, 15-80 mm, 15-70 mm, 15-60 mm, 15-50 mm, 20-90 mm, 20-80 mm, 20-70 mm, 20-60 mm, 20-50 mm, 25-90 mm, 25-80 mm, 25-70 mm, 25-60 mm, or 25-50 mm). In some embodiments, an effective focal length of a first lens (e.g., the lens positioned closest to the pupil plane) is between 10-150 mm (e.g., between 10-140 mm, 10-130 mm, 10-120 mm, 10-110 mm, 10-100 mm, 10-90 mm, 10-80 mm, 10-70 mm, 10-60 mm, 10-50 mm, 15-150 mm, 15-130 mm, 15-120 mm, 15-110 mm, 15-100 mm, 15-90 mm, 15-80 mm, 15-70 mm, 15-60 mm, 15-50 mm, 20-150 mm, 20-130 mm, 20-120 mm, 20-110 mm, 20-100 mm, 20-90 mm, 20-80 mm, 20-70 mm, 20-60 mm, 20-50 mm, 25-150 mm, 25-130 mm, 25-120 mm, 25-110 mm, 25-100 mm, 25-90 mm, 25-80 mm, 25-70 mm, 25-60 mm, 25-50 mm, 30-150 mm, 30-130 mm, 30-120 mm, 30-110 mm, 30-100 mm, 30-90 mm, 30-80 mm, 30-70 mm, 30-60 mm, 30-50 mm, 35-150 mm, 35-130 mm, 35-120 mm, 35-110 mm, 35-100 mm, 35-90 mm, 35-80 mm, 35-70 mm, 35-60 mm, 35-50 mm, 40-150 mm, 40-130 mm, 40-120 mm, 40-110 mm, 40-100 mm, 40-90 mm, 40-80 mm, 40-70 mm, 40-60 mm, 40-50 mm, 45-150 mm, 45-130 mm, 45-120 mm, 45-110 mm, 45-100 mm, 45-90 mm, 45-80 mm, 45-70 mm, 45-60 mm, 45-50 mm, 50-150 mm, 50-130 mm, 50-120 mm, 50-110 mm, 50-100 mm, 50-90 mm, 50-80 mm, 50-70 mm, or 50-60 mm). In some embodiments, for an 8 mm pupil diameter, the lens diameter is 16-24 mm. In some embodiments, for a 7 mm pupil diameter, the lens diameter is 12-20 mm. In some embodiments, the f-number of lens assembly is between 2 and 5. The use of a common lens assembly (e.g., lens assembly 110) in both a wavefront sensor and a corneal topographer allows the integration of the wavefront sensor and the corneal topographer without needing large diameter optics.

Device 100 also includes a wavefront sensor. In some embodiments, the wavefront sensor includes lens assembly 110, first light source 120, an array of lenses 132 (also called herein lenslets), and first image sensor 140. In some embodiments, the wavefront sensor includes additional components.

First light source 120 is configured to emit first light and transfer the first light emitted from the first light source toward eye 170 through lens assembly 110, as depicted in FIG. 1B.

Although several figures illustrated herein (e.g., FIGS. 1A-1C and 1I-1K) include eye 170 and its components (e.g., cornea 172 and lens 174) to illustrate the operations of device 100 with eye 170, eye 170 and its components are not part of a device (e.g., device 100 illustrated in FIGS. 1A-1C, device 102 illustrated in FIGS. 1I-1J, or a device illustrated in FIG. 1K).

Turning back to FIG. 1A, in some embodiments, first light source 120 is configured to emit light of a single wavelength or a narrow band of wavelengths. Exemplary first light source 120 includes a laser (e.g., a laser diode) or a light-emitting diode (LED).

In some embodiments, first light source 120 includes a lens (as shown in FIG. 1A) to change the divergence of the light emitted from first light source 120 so that the light, after passing through lens assembly 110, is collimated.

In some embodiments, first light source 120 includes a pinhole (e.g., having a diameter of 1 mm or less, such as 400 μm, 500 μm, 600 μm, 700 μm, 800 μm, 900 μm, and 1 mm).

Because lens assembly 110 is positioned closer to eye 170 than first light source 120 (e.g., light from first light source 120 passes through lens assembly 110), in some cases, it is important to reduce back reflection of the light at lens assembly. Thus, in some embodiments, an anti-reflection coating is applied on a back surface (and optionally, a front surface) of lens assembly 110 to reduce back reflection. In some embodiments, first light source 120 is configured to transfer the first light emitted from first light source 120 off an optical axis of device 100 (e.g., an optical axis of lens assembly 110), as shown in FIG. 1B (e.g., the first light emitted from first light source 120 propagates parallel to, and offset from, the optical axis of lens assembly 110). This reduces back reflection of the first light emitted from first light source 120, by lens assembly 110, toward first image sensor 140. In some embodiments, the wavefront sensor includes a quarter-wave plate to reduce back reflection, of the first light, from lens assembly 110 (e.g., light reflected from lens assembly 110 is attenuated by the quarter-wave plate). In some embodiments, lens assembly 110 includes a lens that is tilted from an optical axis of the device, to reduce back reflection, of the first light, from the tilted lens (e.g., by reflecting the light toward a direction that does not face first image sensor 140). In some embodiments, a curvature of a lens in lens assembly 110 is selected so that reflection, of the first light, from the lens is directed toward a direction that does not face first image sensor 140.

First image sensor 140 is configured to receive light, from eye 170, transmitted through lens assembly 110 and the array of lenses 132. In some embodiments, the light from eye 170 includes light scattered at a retina or fovea of eye 170 (in response to the first light from first light source 120). For example, as shown in FIG. 1B, light from eye 170 passes multiple optical elements, such as lens assembly 110, beam steerer 122, lens 124, beam steerer 126, mirror 128, and lens 130, and reaches first image sensor 140.

Beam steerer 122 is configured to reflect light from light source 120 and transmit light from eye 170, as shown in FIG. 1B. Alternatively, beam steerer 122 is configured to transmit light from light source 120 and reflect light from eye 170. In some embodiments, beam steerer 122 is a beam splitter (e.g., 50:50 beam splitter, polarizing beam splitter, etc.). In some embodiments, beam steerer 122 is a wedge prism, and when first light source 120 is configured to have a linear polarization, the polarization of the light emitted from first light source 120 is configured to reflect at least partly by the wedge prism. Light of a polarization that is perpendicular to the linear polarization of the light emitted from first light source 120 is transmitted through the wedge prism. In some cases, the wedge prism also reduces light reflected from cornea 172 of eye 170.

In some embodiments, beam steerer 122 is tilted at such an angle (e.g., an angle between the optical axis of device 100 and a surface normal of beam steerer 122 is at an angle less than 45°, such as 30°) so that the space occupied by beam steerer 122 is reduced.

In some embodiments, device 100 includes lenses 124 and 130 to modify a working distance of device 100.

The array of lenses 132 is arranged to focus incoming light onto multiple spots, which are imaged by first image sensor 140. As in Shack-Hartmann wavefront sensor, an aberration in a wavefront causes displacements (or disappearances) of the spots on first image sensor 140. In some embodiments, a Hartmann array is used instead of the array of lenses 132. A Hartmann array is a plate with an array of apertures (e.g., through-holes) defined therein.

In some embodiments, lens 124, lens 130, and the array of lenses 132 are arranged such that the wavefront sensor is configured to measure a reduced range of optical power. A wavefront sensor that is capable of measuring a wide range of optical power may have less accuracy than a wavefront sensor that is capable of measuring a narrow range of optical power. Thus, when a high accuracy in wavefront sensor measurements is desired, the wavefront sensor can be designed to cover a narrow range of optical power. For example, a wavefront sensor for diagnosing low and medium myopia can be configured with a narrow range of optical power between 0 and −6.0 diopters, with its range centering around −3.0 diopters. Although such a wavefront sensor may not provide accurate measurements for diagnosing hyperopia (or determining a prescription for hyperopia), the wavefront sensor would provide more accurate measurements for diagnosing myopia (or determining a prescription for myopia) than a wavefront sensor that can cover both hyperopia and myopia (e.g., from −6.0 to +6.0 diopters). In addition, there are certain populations in which it is preferable to maintain a center of the range at a non-zero value. For example, in some Asian populations, the optical power may range from +6.0 to −14.0 diopters (with the center of the range at −4.0 diopters), whereas in some Caucasian populations, the optical power may range from +8.0 to −12.0 diopters (with the center of the range at −2.0 diopters). The center of the range can be shifted by moving the lenses (e.g., lens 124, lens 130, and/or the array of lenses 132). For example, defocusing light from eye 170 can shift the center of the range.

Device 100 further includes a corneal topographer. In some embodiments, the corneal topographer includes lens assembly 110, second light source 150, and second image sensor 160. In some embodiments, as shown in FIG. 1A, second image sensor 160 is distinct from first image sensor 140. In some embodiments, the wavefront sensor includes additional components.

Second light source 150 is configured to emit second light and transfer the second light emitted from second light source 150 toward eye 170. As shown in FIG. 1C, in some embodiments, second light source 150 is configured to transfer the second light emitted from second light source 150 toward eye 170 without transmitting the second light emitted from second light source 150 through lens assembly 110 (e.g., second light from second light source 150 is directly transferred to eye 170 without passing through lens assembly 110).

In some embodiments, device 100 includes beam steerer 126 configured to transfer light from eye 170, transmitted through lens assembly 110, toward first image sensor 140 and/or second image sensor 160. For example, when device 100 is configured for wavefront sensing (e.g., when light from first light source 120 is transferred toward eye 170), beam steerer 126 transmits light from eye 170 toward first image sensor 140, and when device 100 is configured for corneal topography (e.g., when light from second light source 150 is transferred toward eye 170), beam steerer 126 transmits light from eye 170 toward second image sensor 160.

Second light source 150 is distinct from first light source 120. In some embodiments, first light source 120 and second light source 150 emit light of different wavelengths (e.g., first light source 120 emits light of 900 nm wavelength, and second light source 150 emits light of 800 nm wavelength; alternatively, first light source 120 emits light of 850 nm wavelength, and second light source 150 emits light of 950 nm wavelength). In some embodiments, beam steerer 126 is a dichroic mirror (e.g., a mirror that is configured to transmit the first light from first light source 120 and reflect the second light from second light source 150, or alternatively, reflect the first light from first light source 120 and transmit the second light from second light source 150). In some embodiments, beam steerer 126 is a movable mirror (e.g., a mirror that can flip or rotate to steer light toward first image sensor 140 and second image sensor 160). In some embodiments, beam steerer 126 is a beam splitter. In some embodiments, beam steerer 126 is configured to transmit light of a first polarization and reflect light of a second polarization that is distinct from (e.g., perpendicular to) the first polarization. In some embodiments, beam steerer 126 is configured to reflect light of the first polarization and transmit light of the second polarization.

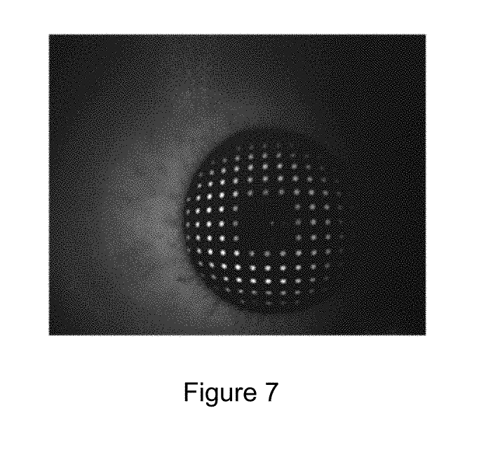

In some embodiments, second light source 150 is configured to project an array of spots on the eye. In some embodiments, the array of spots is arranged in a grid pattern (e.g., FIG. 7). In some embodiments, second light source 150 is configured to project light in a pattern of a plurality of concentric rings (e.g., Placido's disk).

In some embodiments, second light source 150 includes one or more light emitters 152 (e.g., light-emitting diodes) and diffuser 154 (e.g., a diffuser plate having an array of spots). Exemplary embodiments of second light source 150, which are configured to project an array of spots in accordance with some embodiments, are described below with respect to FIGS. 1D-1G.

FIG. 1D illustrates a front view (shown on the left-hand side of FIG. 1D) and a side view (shown on the right-hand side of FIG. 1D) of second light source 150 in accordance with some embodiments. In FIG. 1D, light emitters 152 mounted on mounting plate 166 are placed to face diffuser 154 so that light emitted from light emitters 152 are directed to a face of diffuser 154. Diffuser 154 includes a pattern 162 (e.g., an array of a grid as shown in FIG. 1D), through which light is transmitted (with diffusion). Diffuser 154 also includes portion 164 that blocks transmission of light. Thus, light from light emitters 152 passes through the pattern 162 and has the shape of the pattern 162.

Compared to second light source 150 shown in FIG. 1D, second light sources 150 shown in FIGS. 1E-1G can have less thickness, which allows placement of lens assembly 110 closer to eye 170. The thickness of second light source 150 (and more importantly, the ability to place lens assembly 110 closer to eye 170) is important. The size of a center hole in diffuser 154 needs to be sufficiently small to project light from second light source 150 on a central part of cornea 172. However, if the size of the center hole in diffuser 154 is too small, only a small angle of light will be captured by lens assembly 110, which will reduce the reliability of wavefront sensing. Thus, one solution is to place lens assembly 110 as close toward eye 170, which allows lens assembly 110 to capture more light without actually changing the diameter of the center hole. In addition, placing lens assembly 110 closer toward eye 170 (e.g., as a first optical element to receive light from eye 170) allows capturing more light from eye 170, compared to placing a lens assembly after other optical elements (e.g., after beam steerer 122 or beam steerer 126).

FIG. 1E illustrates a front view (shown on the left-hand side of FIG. 1E) and a side view (shown on the right-hand side of FIG. 1E) of second light source 150 in accordance with some embodiments. In FIG. 1E, light emitters 152 are placed around diffuser 154 so that light from light emitters 152 is not sent directly to the face of diffuser 154. Instead, second light source 150 shown in FIG. 1E includes one or more mirrors (e.g., a conical mirror), which reflect light from second light source 150 toward the face of diffuser 154. Light from second light source 150 after passing through diffuser 154 has the shape of the pattern 162.

FIG. 1F illustrates a front view (shown on the left-hand side of FIG. 1F), a side view (shown in the middle of FIG. 1F), and a cross-sectional view (shown on the right-hand side of FIG. 1F) of second light source 150 in accordance with some embodiments. In FIG. 1F, second light source 150 includes light emitters 152 and diffuser 190. Diffuser 190 includes portion 192 that is transparent (e.g., optically transparent) to light from light emitters 152 and portion 194 that is configured to diffuse light from light emitters 152. Light emitters 152 are placed along a periphery of diffuser 190 so that light emitted from light emitters 152 are transferred toward the periphery of diffuser 190.

FIG. 1G is similar to FIG. 1F, except that light emitters 152 are arranged on round mounting plate 166 instead of square mounting plate 166.

Although diffusers 154 and 190 are each illustrated as a single component, in some embodiments, a diffuser includes multiple components (or multiple layers). For example, in some embodiments, a diffuser includes a diffusion layer configured to diffuse, spread out, or scatter light, and a separate masking layer for transmitting light in a particular pattern. The diffusion layer can be made from ground glass and/or light scattering material, such as photopolymer and/or polytetrafluoroethylene.

Turning back to FIG. 1A, second image sensor 160 is configured to receive light, from eye 170, transmitted through lens assembly 110. In some embodiments, the light from eye 170 includes light reflected from cornea 172 of eye 170 (in response to the second light from second light source 150). For example, as shown in FIG. 1C, light from eye 170 (e.g., light reflected from cornea 172) passes multiple optical elements, such as lens assembly 110, beam steerer 122, lens 124, beam steerer 126, and lenses 156 and 158, and reaches second image sensor 160.

The lenses in the corneal topographer (e.g., lens assembly 110 and lenses 124, 156, and 158) are configured to image a pattern of light projected on cornea 172 onto second image sensor 160. For example, when an array of spots is projected on cornea 172, the image of the array of spots detected by second image sensor 160 is used to determine the topography of cornea 172 (e.g., a profile of a surface of cornea 172 or a curvature of cornea 172).

FIG. 1H illustrates device 100 in accordance with some embodiments. In FIG. 1H, device 100 includes eyecup 196. In some embodiments, eyecup 196 is configured to position the eye relative to the device. For example, eyecup 196 is configured to be placed against an orbit of the eye so that the eye is positioned for optical measurements, such as wavefront sensing and/or corneal topography measurements. Alternatively, in some embodiments, eyecup 196 is configured to block ambient light (e.g., with or without mechanically positioning the eye relative to device 100.

FIGS. 1I-1J illustrate optical components of portable device 102 in accordance with some embodiments. FIG. 1I is a side view of a portable device that is capable of wavefront sensing. FIG. 1J is a top view of the portable device shown in FIG. 1I.

Device 102 includes a wavefront sensor. In some embodiments, the wavefront sensor includes first light source 120 configured to emit first light (e.g., an infrared light) and transfer the first light emitted from first light source 120 toward eye 170. For example, the first light emitted from first light source 120 is reflected by reflector (e.g., mirror) 134 and beam steerer 122.

Light from eye 170 is transmitted through beam steerer 122 and further through lens 124, lens 156, and lens 158. In some embodiments, additional lenses or fewer lenses are used. In some embodiments, an aperture stop is placed between lens 124 and lens 156.

Beam steerer 126 reflects infrared light toward an array of lenses 132 and transmits visible light toward second image sensor 160 (which operates as a pupil camera). The array of lenses 132 focus the light into a plurality of spots, which are imaged by first image sensor 140. Alternatively, beam steerer 126 transmits infrared light toward an array of lenses 132 and reflects visible light toward second image sensor 160 (when the positions of the array of lenses 132 and second image sensor 160 are exchanged from those illustrated in FIG. 1J).

Although FIGS. 1I-1J illustrate optical components for wavefront sensing, the portable device may include additional components, such as optical components for corneal topography. In some embodiments, the portable device does not include optical components for corneal topography.

FIG. 1K is a schematic diagram illustrating a portable device including a display device in accordance with some embodiments.

In FIG. 1K, the portable device includes display device 136 and one or more lenses 138. Light from display device 136 includes a linearly polarized light (or a component of light having a linear polarization), which is transmitted through one or more lenses 138, polarization sensitive beam splitter 144, and quarter-wave plate 146, gets reflected from mirror 148 (e.g., a refracting mirror or a planar mirror), and pass through quarter-wave plate 146 again. The double-pass through quarter-wave plate 146 rotates the polarization of the light 90°. The rotated linearly polarized light is reflected by polarization sensitive beam splitter 144 toward eye 170.

In some embodiments, one or more lenses 138 include one or more adjustable lenses (e.g., one or more lenses are configured to move so that a focus or a focal length of one or more lenses 138 can be adjusted). In some embodiments, one or more lenses 138 include an adaptive optics (e.g., electro-optic lens) for adjusting a focal length of the adaptive optics.

The portable device also includes a wavefront sensor (e.g., a wavefront sensor in device 102). In some embodiments, the light from the wavefront sensor is an infrared light. The light from the wavefront sensor is reflected by wavelength sensitive mirror 176 toward polarization sensitive beam splitter 144. In some embodiments, polarization sensitive beam splitter 144 is configured to transmit an infrared light so that the light from the wavefront sensor is transmitted toward eye 170 and a reflection of the infrared light from eye 170 is transmitted back through polarization sensitive beam splitter 144 toward wavelength sensitive mirror 176, which reflects the infrared light back toward the wavefront sensor. A visible ambient light is transmitted through wavelength sensitive mirror 176 toward polarization sensitive beam splitter 144. At least a portion of the visible ambient light is transmitted through polarization sensitive beam splitter 144 toward eye 170.

In some embodiments, the portable device includes one or more mirrors to steer the light (e.g., mirror 142 is located between lens 138 and polarization sensitive beam splitter 144 along an optical path from display device 136, mirror 178 is located between device 102 and wavelength sensitive mirror 176).

Although in FIG. 1K, the portable device that operates for a single eye is illustrated, a combination of two portable devices or an integrated portable device with two wavefront sensors and two display devices can be used with two eyes.

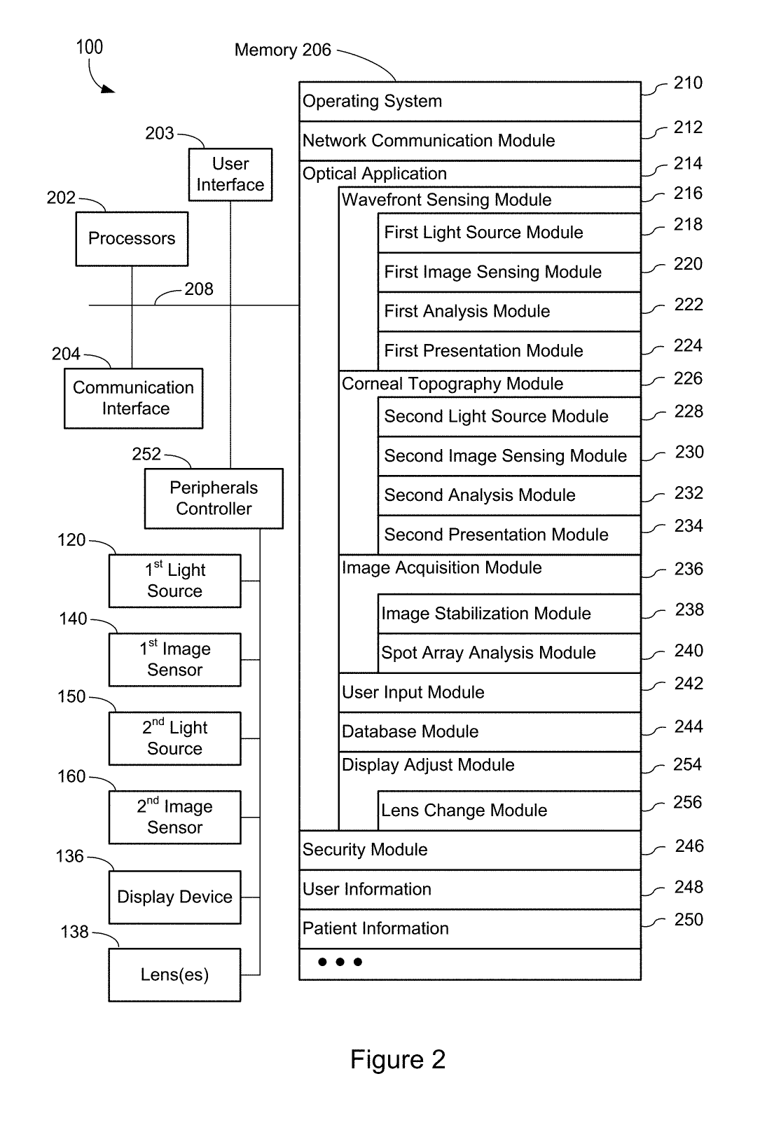

FIG. 2 is a block diagram illustrating electronic components of device 100 in accordance with some embodiments. Device 100 typically includes one or more processing units 202 (central processing units, application processing units, application-specific integrated circuit, etc., which are also called herein processors), one or more network or other communications interfaces 204, memory 206, and one or more communication buses 208 for interconnecting these components. In some embodiments, communication buses 208 include circuitry (sometimes called a chipset) that interconnects and controls communications between system components. In some embodiments, device 100 includes a user interface 203 (e.g., a user interface having a display device, which can be used for displaying acquired images, one or more buttons, and/or other input devices). In some embodiments, device 100 also includes peripherals controller 252, which is configured to control operations of other electrical components of device 100, such as first light source 120, first image sensor 140, second light source 150, second image sensor 160 (e.g., initiating respective light sources to emit light, and/or receiving information, such as images, from respective image sensors), display device 136, and/or one or more lenses 138.

In some embodiments, communications interfaces 204 include wired communications interfaces and/or wireless communications interfaces (e.g., Wi-Fi, Bluetooth, etc.).

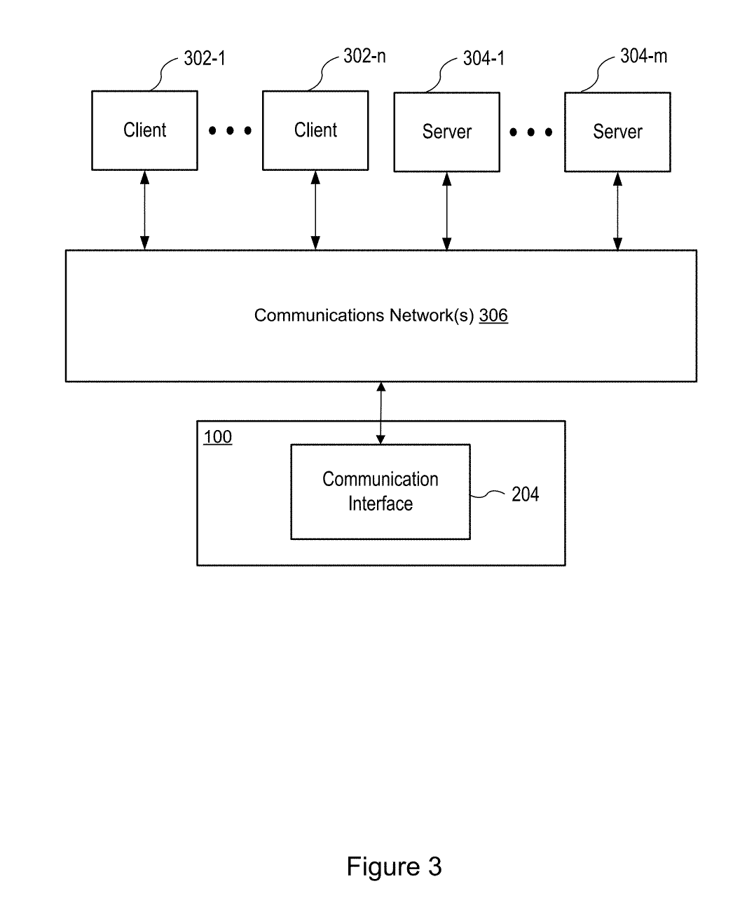

Memory 206 of device 100 includes high-speed random access memory, such as DRAM, SRAM, DDR RAM or other random access solid state memory devices; and may include non-volatile memory, such as one or more magnetic disk storage devices, optical disk storage devices, flash memory devices, or other non-volatile solid state storage devices. Memory 206 may optionally include one or more storage devices remotely located from the processors 202. Memory 206, or alternately the non-volatile memory device(s) within memory 206, comprises a computer readable storage medium (which includes a non-transitory computer readable storage medium and/or a transitory computer readable storage medium). In some embodiments, memory 206 includes a removable storage device (e.g., Secure Digital memory card, Universal Serial Bus memory device, etc.). In some embodiments, memory 206 or the computer readable storage medium of memory 206 stores the following programs, modules and data structures, or a subset thereof:
operating system 210 that includes procedures for handling various basic system services and for performing hardware dependent tasks;
network communication module (or instructions) 212 that is used for connecting device 100 to other computers (e.g., clients 302 and/or servers 304 shown in FIG. 3) via one or more communications interfaces 204 and one or more communications networks 306 (FIG. 3), such as the Internet, other wide area networks, local area networks, metropolitan area networks, and so on;
optical application 214 that controls operations of the light sources and the image sensors; and
security module 246 that protects data stored on device 100 during its storage on device 100 and/or transmission to and from another computer (e.g., clients 302 and/or servers 304); for example, security module 246 may include an encryption module for encrypting data stored on device 100, a decryption module for decrypting encrypted data, either stored on device 100 or received from another computer, and an authentication module for authenticating a user of device 100 and/or a remote computer for communication with device 100 (e.g., for sending and/or receiving data).

In some embodiments, memory 206 also includes one or both of:
user information 248 (e.g., information necessary for authenticating a user of device 100); and
patient information 250 (e.g., optical measurement results and/or information that can identify patients whose optical measurement results are stored on device 100).

In some embodiments, optical application 214 includes the following programs, modules and data structures, or a subset or superset thereof:
wavefront sensing module 216 configured for operating the wavefront sensor in device 100;
corneal topography module 226 configured for operating the corneal topographer in device 100;
image acquisition module 236 configured for analyzing images collected by respective image sensors of device 100;
user input module 242 configured for handling user inputs on device 100 (e.g., pressing of buttons of device 100, etc.);
database module 244 configured to assist storage of data on device 100 and retrieval of data from device 100 (in some embodiments, database module 244 operates in conjunction with security module 246); and
display adjust module 254 configured to adjust one or more lenses.

In some embodiments, wavefront sensing module 216 includes the following programs and modules, or a subset or superset thereof:
first light source module 218 configured for initiating first light source 120 (through peripherals controller 252) to emit light;
first image sensing module 220 configured for receiving images from first image sensor 140;
first analysis module 222 configured for analyzing images received from first image sensor 140; and
first presentation module 224 configured for presenting measurement and analysis results from first analysis module 222 (e.g., graphically displaying images received from first image sensor 140, presenting aberrations shown in images received from first image sensor 140, sending the results to another computer, etc.).

In some embodiments, corneal topography module 226 includes the following programs and modules, or a subset or superset thereof:
second light source module 228 configured for initiating second light source 150 (through peripherals controller 252) to emit light;
second image sensing module 230 configured for receiving images from second image sensor 160;
second analysis module 232 configured for analyzing images received from second image sensor 160; and
second presentation module 234 configured for presenting measurement and analysis results from second analysis module 232 (e.g., graphically displaying images received from second image sensor 160, presenting cornea curvatures determined from images received from second image sensor 160, sending the results to another computer, etc.).

In some embodiments, corneal topography module 226 includes instructions for determining a vergence of an eye from one or more images received by second image sensing module 230.

In some embodiments, image acquisition module 236 includes the following programs and modules, or a subset or superset thereof:
image stabilization module 238 configured for reducing blurring during acquisition of images by image sensors; and
spot array analysis module 240 configured for analyzing spot arrays (e.g., measuring displacements and/or disappearances of spots in the spot arrays).

In some embodiments, display adjust module 254 includes the following programs and modules, or a subset or superset thereof:
lens change module 256 for adjusting (e.g., moving) one or more lenses (e.g., through peripherals controller 252);
a first comparison module for comparing a refraction value with a reference value (e.g., a reference value stored in patient information 250); and
a second comparison module for comparing a refraction value with a limit value (e.g., a limit value stored in patient information 250).

In some embodiments, first image sensing module 220 initiates execution of image stabilization module 238 to reduce blurring during acquisition of images by first image sensor 140, and second image sensing module 230 initiates execution of image stabilization module 238 to reduce blurring during acquisition of images by second image sensor 160.

In some embodiments, first analysis module 222 initiates execution of spot array analysis module 240 to analyze spot arrays in images acquired by first image sensor 140, and second analysis module 232 initiates execution of spot array analysis module 240 to analyze spot arrays in images acquired by second image sensor 160.

Each of the above identified modules and applications correspond to a set of instructions for performing one or more functions described above. These modules (i.e., sets of instructions) need not be implemented as separate software programs, procedures or modules, and thus various subsets of these modules may be combined or otherwise re-arranged in various embodiments. In some embodiments, memory 206 may store a subset of the modules and data structures identified above. Furthermore, memory 206 may store additional modules and data structures not described above.

Although device 100 is described above with respect to FIG. 2, device 102 also includes one or more components described with respect to FIG. 2. For brevity, such details are not repeated herein.

FIG. 3 is a block diagram illustrating a distributed computing system in accordance with some embodiments. In FIG. 3, the distributed computing system includes one or more client computers 302, one or more server systems 304, communications network 306, and device 100.

Client computers 302 can be any of a number of computing devices (e.g., Internet kiosk, personal digital assistant, cell phone, smart phone, gaming device, desktop computer, laptop computer, handheld computer, or combinations thereof) used to enable the activities described below. Client computer(s) 302 is also referred to herein as client(s). Client 302 typically includes a graphical user interface (GUI). In some embodiments, client 302 is connected to device 100 via communications network 106. As described in more detail below, the graphical user interface is used to display results from device 100 (e.g., acquired images and/or analysis results). In some embodiments, one or more clients are used to perform the analysis (for example, when device 100 does not include sufficient computational capabilities, images can be sent to one or more clients for analysis).

In some embodiments, the distributed computing system includes one or more server systems (also called server computers) 304 connected to communications network 306. One or more server systems 304 store results from device 100 (and a plurality of similar devices). For example, one or more server systems 304 store images transmitted from device 100 and/or analysis results. In some embodiments, one or more server systems 304 provide the stored images and/or analysis results to one or more clients (e.g., computers used by medical professionals) 302. In some embodiments, one or more server systems 304 are used to perform the analysis (e.g., the one or more servers analyze images sent by device 100).

In some embodiments, communications networks 306 are the Internet. In other embodiments, the communications networks 306 can be any local area network (LAN), wide area network (WAN), metropolitan area network, or a combination of such networks. In some embodiments, communications networks 306 include a wired network and/or a wireless network (e.g., Wi-Fi, Bluetooth, etc.).

In some embodiments, device 100 receives one or more software applications or one or more software modules from one or more server systems 304 or one or more clients 302 (e.g., using the wired communication network and/or the wireless communication network).

Notwithstanding the discrete blocks in FIGS. 2 and 3, these figures are intended to be a functional description of some embodiments, although, in some embodiments, the discrete blocks in FIGS. 2 and 3 can be a structural description of functional elements in the embodiments. One of ordinary skill in the art will recognize that an actual implementation might have the functional elements grouped or split among various components. In practice, and as recognized by those of ordinary skill in the art, items shown separately could be combined and some items could be separated. For example, in some embodiments, security module 246 is part of optical application 214. In other embodiments, wavefront sensing module 216 and corneal topography module 226 are implemented as separate applications.

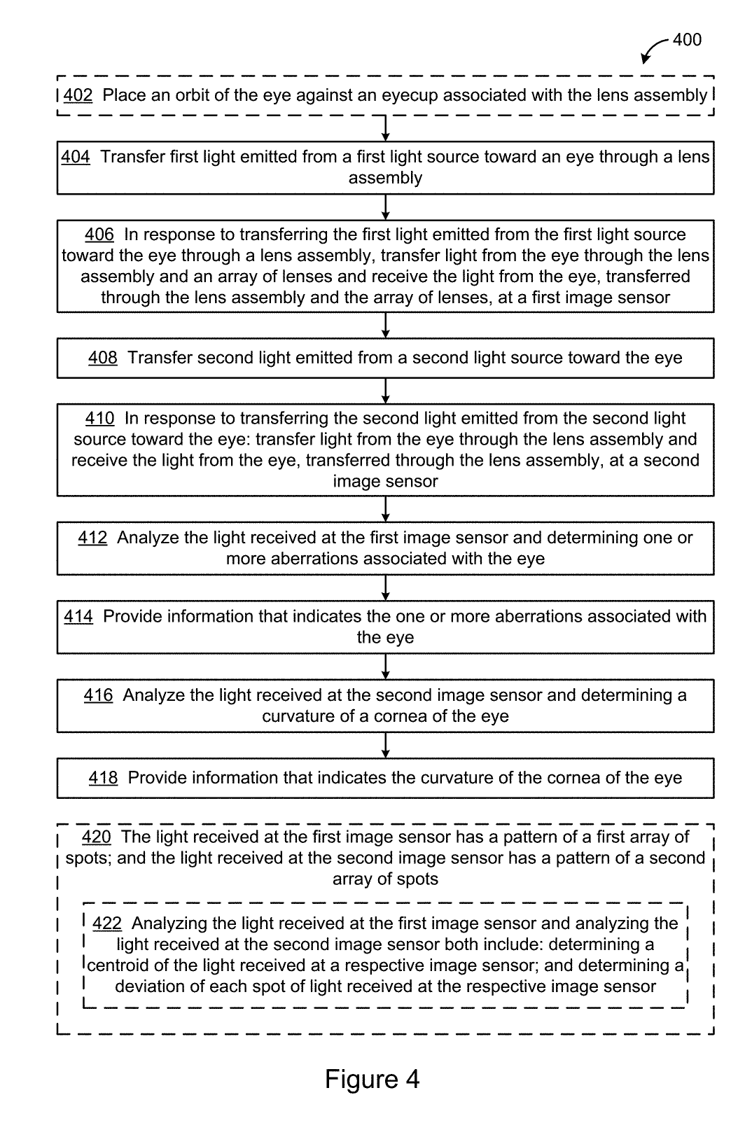

FIG. 4 is a flowchart representing method 400 for optical measurements (e.g., wavefront sensing and keratometry (or corneal topography)) with a portable device, in accordance with some embodiments.

In some embodiments, method 400 includes (402) placing an orbit of the eye against an eyecup (e.g., eyecup 196 in FIG. 1H) associated with the lens assembly. In some embodiments, the eyecup blocks ambient light, which helps the pupil of the eye to dilate for more accurate wavefront sensing.

Method 400 includes (404) transferring first light emitted from a first light source toward the eye through a lens assembly, and, in response to transferring the first light emitted from the first light source toward the eye through a lens assembly, (406) transferring light from the eye through the lens assembly and an array of lenses; and receiving the light from the eye, transferred through the lens assembly and the array of lenses, at a first image sensor. For example, as shown in FIG. 1B, first light emitted from first light source 120 is transferred toward eye 170 through lens assembly 110. In response, light from eye 170 (e.g., light scattered and/or reflected from inside eye 170) is transferred through lens assembly 110 and the array of lenses 132, and is received at first image sensor 140.

In some embodiments, receiving the light from the eye at the first image sensor includes acquiring multiple images of the light from the eye with the first image sensor (e.g., multiple images are taken in a few seconds, or even in less than a second).

Method 400 also includes (408) transferring second light emitted from a second light source toward the eye, and, in response to transferring the second light emitted from the second light source toward the eye, (410) transferring light from the eye through the lens assembly; and receiving the light from the eye, transferred through the lens assembly, at a second image sensor. For example, as shown in FIG. 1C, second light emitted from second light source 150 is transferred toward eye 170. In response, light from eye 170 (e.g., light scattered and/or reflected from cornea 172 of eye 170) is transferred through lens assembly 110, and received at second image sensor 160. In some embodiments, receiving the light from the eye at the second image sensor includes acquiring multiple images of the light from the eye with the second image sensor.

Method 400 further includes (412) analyzing the light received at the first image sensor and determining one or more aberrations associated with the eye. For example, displacements and/or disappearances of spots in the image received at first image sensor 140 are measured and used to determine one or more aberrations associated with eye 170.

Method 400 includes (414) providing information that indicates the one or more aberrations associated with the eye. For example, a spherical aberration and an astigmatism of the eye (e.g., in diopter) can be reported.

Method 400 includes (416) analyzing the light received at the second image sensor and determining a curvature of a cornea of the eye; and (418) providing information that indicates the curvature of the cornea of the eye. In some embodiments, method 400 includes determining a corneal topography of the eye (e.g., determining a profile of the cornea of the eye). In some embodiments, method 400 includes determining the curvature of the cornea of the eye from the corneal topography of the eye. In some embodiments, method 400 includes determining two curvatures of the cornea (e.g., flat radius and steep radius) and providing information that indicates both curvatures of the cornea. In some embodiments, method 400 includes providing information that indicates a difference between the two curvatures and an angle of a respective radius with respect to a reference axis of the eye (e.g., a horizontal axis or a vertical axis). In some embodiments, method 400 includes providing information that indicates an average of the two curvatures.

In some embodiments, the light received at the first image sensor has (420) a pattern of a first array of spots; and the light received at the second image sensor has a pattern of a second array of spots. For example, the light received at first image sensor 140 has a pattern of an array of spots, because of the array of lenses 132 (e.g., each lens in the array of lenses 132 is responsible for a single spot on first image sensor 140). The light received at second image sensor 160 generally has a pattern of light projected on cornea 172 of eye 170 (e.g., FIG. 7). Unlike conventional corneal topographers, which utilize a pattern of concentric rings, a pattern of an array of spots can be projected on cornea 172 of eye 170, and the light received at second image sensor 160 also has a pattern of an array of spots. The use of an array of spots enables images acquired by second image sensor 160 to be analyzed in a similar manner as images acquired by first image sensor 140. In addition, it has been found that the use of a pattern of an array of spots for corneal topography further improves an accuracy of the corneal topography. Because an array of spots provides more discrete points to track, compared to conventional concentric rings, the resolution of corneal topography can be further improved with the use of a pattern of an array of spots.

In some embodiments, analyzing the light received at the first image sensor and analyzing the light received at the second image sensor both include (422): determining a centroid of the light received at a respective image sensor; and determining a deviation of each spot of light received at the respective image sensor. Thus, deviations (or displacements) of the spots are used to determine aberrations (in case of wavefront sensing) and/or deformations of the cornea (in case of corneal topography).

It should be understood that the particular order in which the operations in FIG. 4 have been described is merely exemplary and is not intended to indicate that the described order is the only order in which the operations could be performed. One of ordinary skill in the art would recognize various ways to reorder the operations described herein. For example, the analyzing operation (412) may be performed before the transferring operation (408). In another example, the analyzing operation (416) may be performed in conjunction with the analyzing operation (412), before the providing operation (414). Additionally, it should be noted that details of other processes described herein with respect to method 500 described herein are also applicable in an analogous manner to method 400 described above with respect to FIG. 4. For example, the transferring, receiving, and analyzing operations, described above with reference to method 400 optionally have one or more of the characteristics of the transferring, receiving, and analyzing operations described herein with reference to method 500 described herein. For brevity, these details are not repeated here.

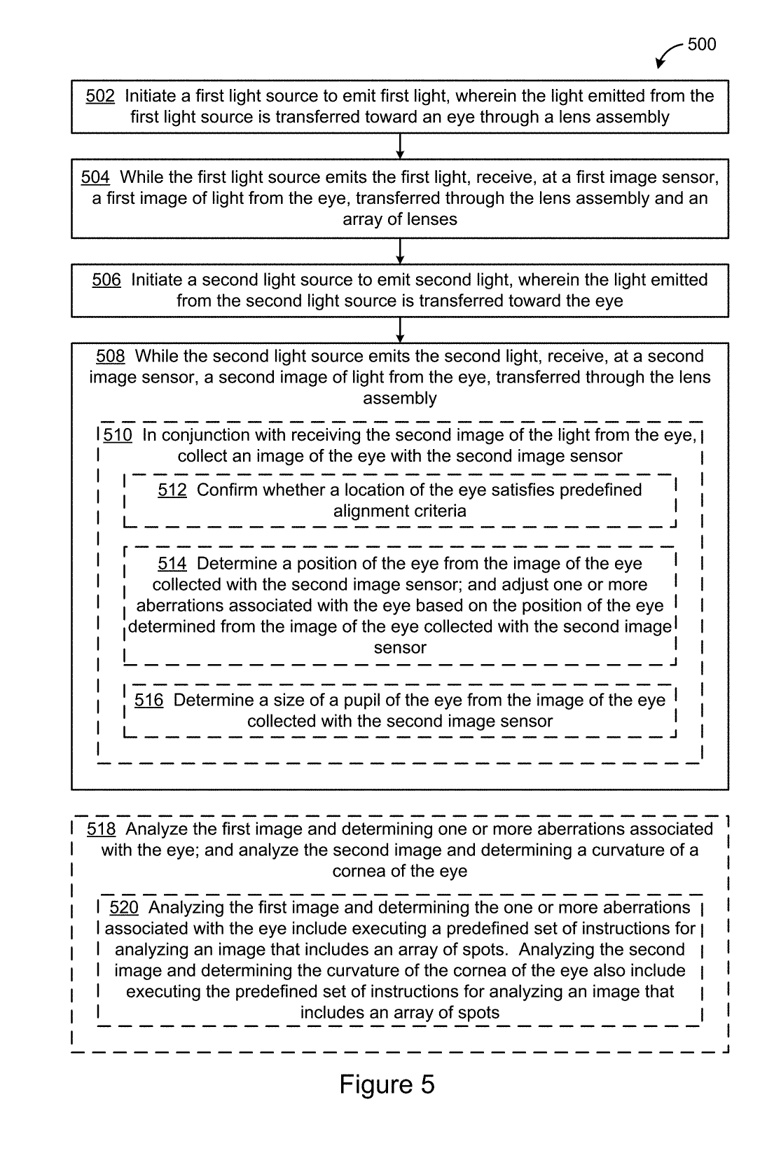

FIG. 5 is a flowchart representing method 500 of optical measurements (e.g., wavefront sensing and keratometry (or corneal topography)) with a portable device, in accordance with some embodiments.

Method 500 is performed at an electronic device (e.g., device 100) that includes one or more processors (e.g., processors 202, FIG. 2) and memory (e.g., memory 206, FIG. 2) storing instructions for execution by the one or more processors.

Method 500 includes (502) initiating a first light source to emit first light (e.g., using first light source module 218 to initiate first light source 120 to emit first light). The first light emitted from the first light source is transferred toward an eye through a lens assembly. For example, as shown in FIG. 1B, the first light emitted from first light source 120 is transferred toward eye 170 through lens assembly 110.

Method 500 includes, while the first light source emits the first light, (504) receiving, at a first image sensor, a first image of light from the eye, transferred through the lens assembly and an array of lenses (e.g., using first image sensing module 220).

Method 500 includes (506) initiating a second light source to emit second light (e.g., using second light source module 228 to initiate second light source 150 to emit second light). The light emitted from the second light source is transferred toward the eye. For example, as shown in FIG. 1C, the second light emitted from second light source 10 is transferred toward eye 170.

Method 500 includes (508), while the second light source emits the second light, receiving, at a second image sensor, a second image of light from the eye, transferred through the lens assembly (e.g., using second image sensing module 230).

In some embodiments, method 500 includes, in conjunction with receiving the second image of the light from the eye, (510) collecting an image of the eye with the second image sensor. For example, an image of the eye is acquired with second image sensor 160. This image can be used to determine whether the eye is properly positioned for optical measurements (e.g., wavefront sensing and/or corneal topography). In some embodiments, the image of the eye is collected with the second image sensor in temporal proximity to receiving the second image of the light from the eye. This reduces any error due to the movement of the eye between collecting the image of the eye and receiving the second image. For example, the image of the eye is collected with the second image sensor immediately before receiving the second image of the light from the eye. Alternatively, the image of the eye is collected with the second image sensor immediately after receiving the second image of the light from the eye. In some embodiments, method 500 includes providing the image of the eye for display to a user and receiving a user input (e.g., pressing on a “go” or “acquire” button) to initiate receiving the second image.

In some embodiments, method 500 includes (512) confirming whether a location of the eye satisfies predefined alignment criteria. For example, method 500 includes determining that the eye is offset from the center of the image by more than a distance, and in response, providing a warning (e.g., either a visible or audible warning to indicate that the second image may not be usable or the result may not be accurate) and/or preventing receiving of the second image.

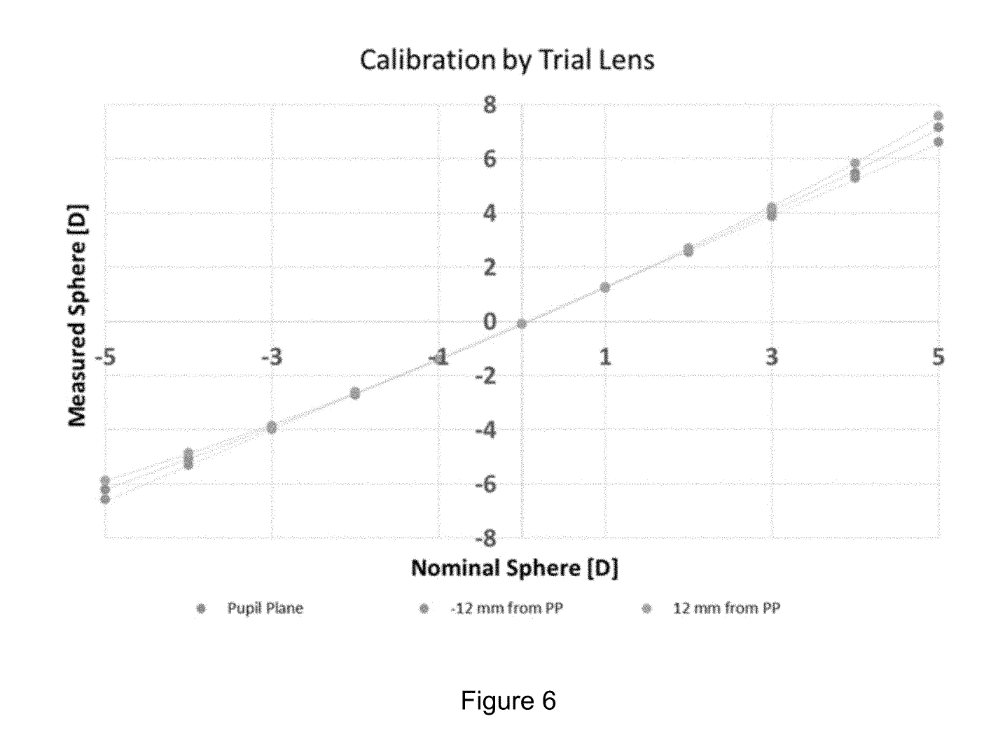

In some embodiments, method 500 includes (514) determining a position of the eye from the image of the eye collected with the second image sensor; and adjusting one or more aberrations associated with the eye based on the position of the eye determined from the image of the eye collected with the second image sensor. The inventors of this application have found that the measurement of the power of the eye is incorrect if the eye is placed away from a pupil plane of device 100. The inventors of this application have also discovered that the error can be corrected if the distance from the eye to the pupil plane of device 100 is known. FIG. 6 illustrates exemplary calibration curves that can be used to calibrate the measurements. For example, if the eye is positioned away from the pupil plane of device 100 by 12 mm, the measured power of the eye may be off by approximately 10%. Thus, the measured power of the eye should be adjusted accordingly.

In some embodiments, method 500 includes (516) determining a size of a pupil of the eye from the image of the eye collected with the second image sensor. This allows a user of device 100 to ensure that the pupil size is sufficient to measure high order aberrations, because high order aberrations are difficult to measure if the pupil size is not sufficiently large.

In some embodiments, method 500 includes: (518) analyzing the first image and determining one or more aberrations associated with the eye (e.g., determining spherical aberrations and astigmatism of the eye); and analyzing the second image and determining a curvature of a cornea of the eye. In some embodiments, determining the curvature of the cornea of the eye includes determining a corneal topography of the eye.

In some embodiments, the instructions include a predefined set of instructions for analyzing an image that includes an array of spots (e.g., spot array analysis module 240 in FIG. 2). Analyzing the first image and determining the one or more aberrations associated with the eye include (520) executing the predefined set of instructions for analyzing an image that includes an array of spots; and analyzing the second image and determining the curvature of the cornea of the eye also include executing the predefined set of instructions for analyzing an image that includes an array of spots. Because the same predefined set of instructions is used for analyzing both images received at the first image sensor and at the second image sensor, the software application can be made smaller, faster, and more efficient.

It should be understood that the particular order in which the operations in FIG. 5 have been described is merely exemplary and is not intended to indicate that the described order is the only order in which the operations could be performed. One of ordinary skill in the art would recognize various ways to reorder the operations described herein. Additionally, it should be noted that details of other processes described herein with respect to method 400 described herein are also applicable in an analogous manner to method 500 described above with respect to FIG. 5. For example, the transferring, receiving, and analyzing operations, described above with reference to method 500 optionally have one or more of the characteristics of the transferring, receiving, and analyzing operations described herein with reference to method 400 described herein. For brevity, these details are not repeated here.

FIG. 6 illustrates exemplary calibration curves for adjusting one or more aberrations of an eye based on a position of the eye relative to the device, in accordance with some embodiments. In FIG. 6, each curve represents measured spherical powers of simulated eyes (e.g., simulated by representative lenses of known powers) as functions of their true (nominal) spherical powers. The curves shown in FIG. 6 also indicate that the measured spherical powers of the simulated eyes vary depending on the position of the eye. As explained above, if the eye is positioned away from the pupil plane of device 100 by 12 mm, the measured power of the eye can be off by as much as 10%. By using the calibration curves shown in FIG. 6, the true spherical power of an eye can be determined. Furthermore, the error caused by the position of the eye can be reduced.

FIG. 7 is an exemplary image of an eye with projection of a spot array pattern in accordance with some embodiments. As shown in FIG. 7, the sport array pattern has a shape of a grid, unlike concentric circles used in conventional Placido corneal topographers. As described above, the use of the spot array pattern improves the accuracy of corneal topography, and also improves the processing of images for corneal topography by portable devices, because the same set of instructions can be used for analyzing both images for wavefront sensing and images for corneal topography. The details of using the spot array pattern, which are described above, are not repeated here.

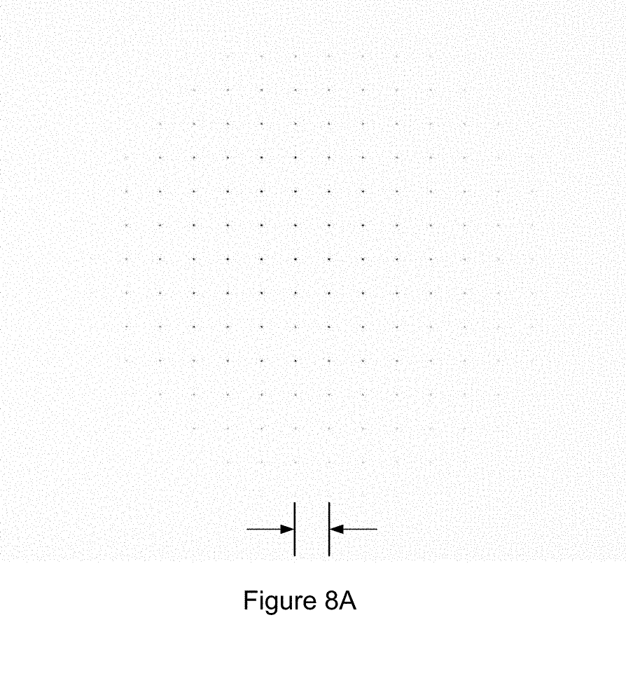

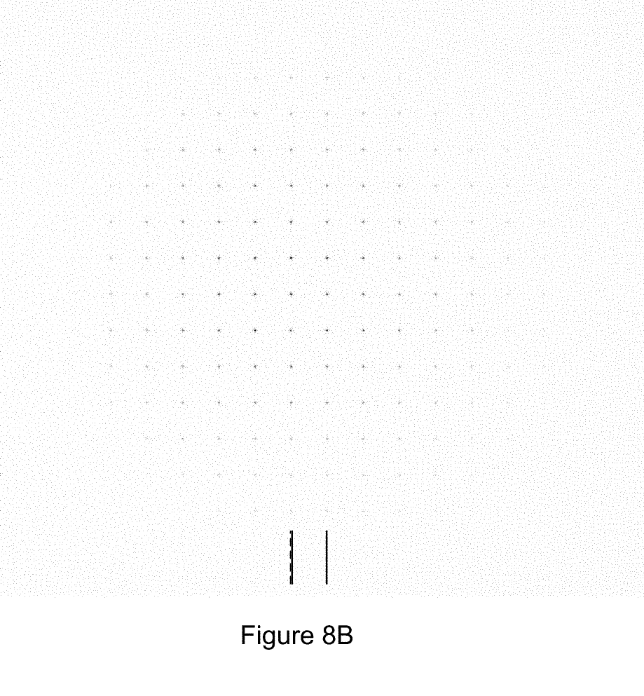

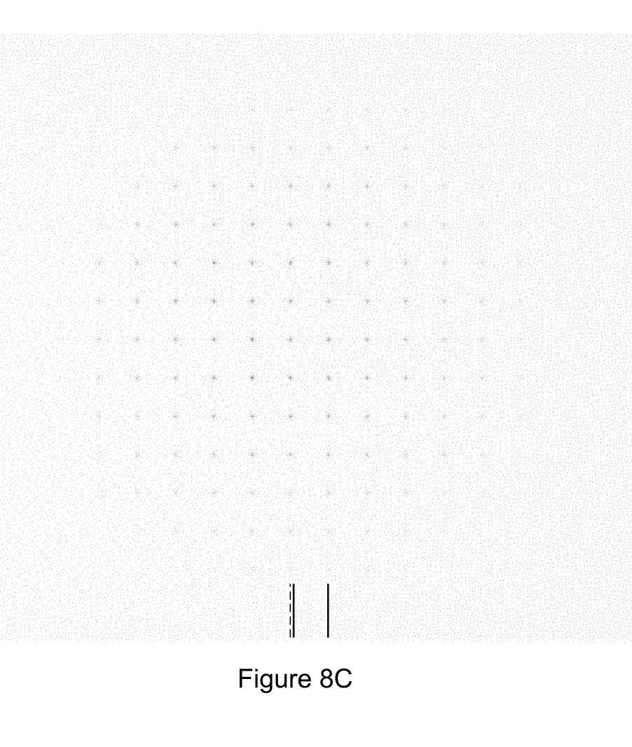

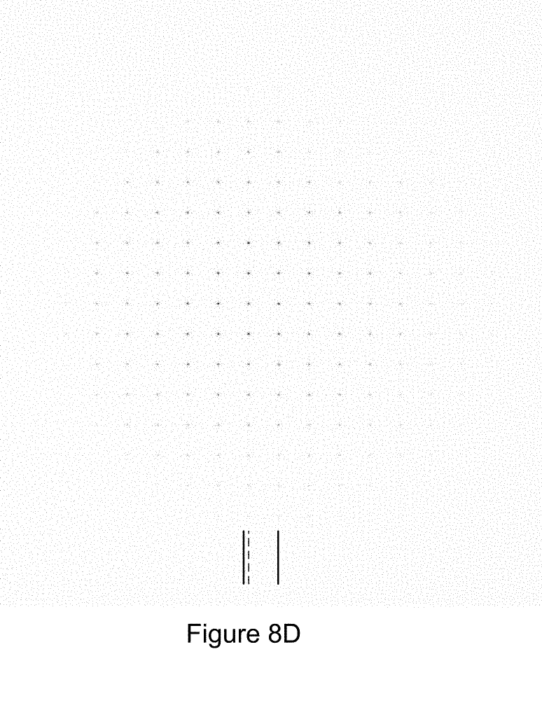

FIGS. 8A-8D illustrate examples of images collected by a wavefront sensor for eyes having different refraction values.

FIG. 8A illustrates an image collected by a wavefront sensor (e.g., image sensor 140 in FIG. 1J) from an eye having a refraction value of a zero diopter. The spacing of an array of spots is indicated with two parallel solid lines at a bottom of the image.

FIG. 8B illustrates an image collected by a wavefront sensor (e.g., image sensor 140 in FIG. 1J) from an eye having a refraction value of +5 diopter (e.g., an eye with hyperopia). The spacing of an array of spots is indicated with a dashed line at a bottom of the image. The two parallel solid lines representing the spacing of spots from an eye having a refraction value of a zero diopter are also shown to highlight the changes. As shown in FIG. 8B, the spacing of the array of spots increases with the increased refraction value.

FIG. 8C illustrates an image collected by a wavefront sensor (e.g., image sensor 140 in FIG. 1J) from an eye having a refraction value of +10 diopter (e.g., an eye with hyperopia). The spacing of an array of spots is indicated with a dashed line at a bottom of the image. The two parallel solid lines representing the spacing of spots from an eye having a refraction value of a zero diopter are also shown to highlight the changes. FIG. 8C shows that the spacing of the array of spots has increased further from the spacing illustrated in FIGS. 8A and 8B.

FIG. 8D illustrates an image collected by a wavefront sensor (e.g., image sensor 140 in FIG. 1J) from an eye having a refraction value of −8 diopter (e.g., an eye with myopia). The spacing of an array of spots is indicated with a dashed line at a bottom of the image. The two parallel solid lines representing the spacing of spots from an eye having a refraction value of a zero diopter are also shown to highlight the changes. As shown in FIG. 8D, the spacing of the array of spots decreases with the decreased refraction value.

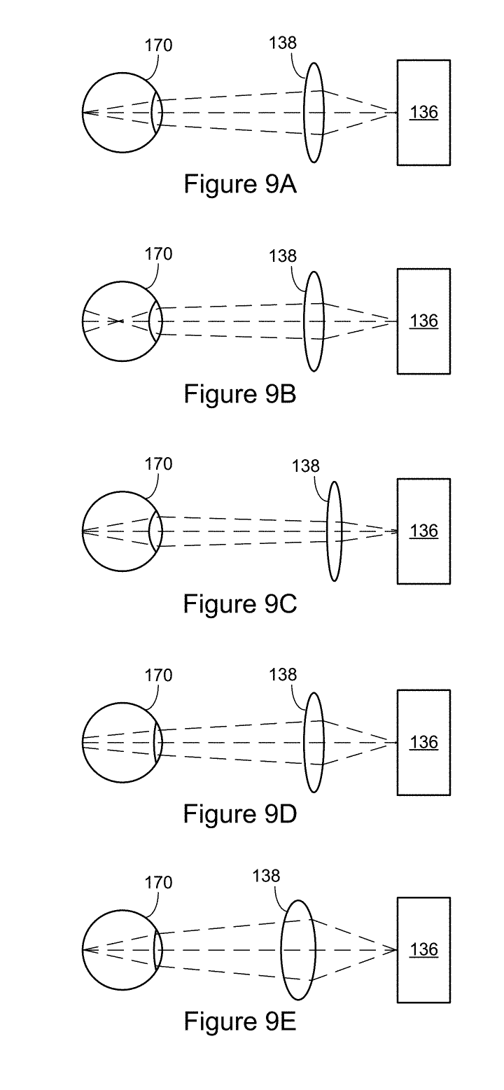

Thus, the wavefront sensor can measure a refraction value of an eye, which is used to adjust a display device as described below with respect to FIGS. 9A-9E.

FIGS. 9A-9E are schematic diagrams illustrating operations of a portable electronic device with a display device in accordance with some embodiments.

FIG. 9A illustrates display device 136 with lens 138, where an image on display device 136 is projected toward eye 170 through lens 138. As shown in FIG. 9A, the image on display device 136 is imaged by lens 138 and a lens of eye 170 so that the image is focused on a retina of eye 170. As used herein, a refraction value of eye 170 refers to a refraction value of an eye lens of eye 170 (e.g., a focal length of eye 170 refers to a focal length of the eye lens of eye 170).

FIG. 9B illustrates that the refraction value of eye 170 has changed (e.g., the refractive power of eye 170 and/or the eye lens has increased). As a result, the image on display device 136 is not focused on the retina of eye 170.

FIG. 9C illustrates that, based on the refraction value of the eye 170 (e.g., the refraction value of the eye lens, which is different from lens 138), lens 138 is adjusted. For example, the position of lens 138 is changed and/or the power of lens 138 is changed (e.g., the position of lens 138 is changed without changing the power of lens 138, the power of lens 138 is changed without changing the position of lens 138, or both the position of lens 138 and the power of lens 138 are changed). When the refraction value of eye 170 increases (e.g., the focal length of eye 170 and/or the eye lens decreases), the position of lens 138 is changed (e.g., lens 138 moves closer to display 136) without changing the power of lens 138, the power of lens 138 is changed (e.g., the power of lens 138 decreases) without moving the position of lens 138, or the position of lens 138 is changed and the power of lens 138 is changed (e.g., lens 138 moves toward display 136 and the power of lens 138 decreases, lens 138 moves away from display 136 and the power of lens 138 decreases, or lens 138 moves toward display 136 and the power of lens 138 increases). In FIG. 9C, the power of lens 138 is decreased and lens 138 is moved toward display device 136.

FIG. 9D illustrates that the refraction value of eye 170 has changed (e.g., the refractive power of eye 170 or the eye lens has decreased so that the focal length of the eye lens has increased). As a result, the image on display device 136 is not focused on the retina of eye 170.

FIG. 9E illustrates that, based on the refraction value of eye 170, lens 138 is adjusted. For example, the position of lens 138 is changed and/or the power of lens 138 is changed (e.g., the position of lens 138 is changed without changing the power of lens 138, the power of lens 138 is changed without changing the position of lens 138, or both the position of lens 138 and the power of lens 138 are changed). When the refraction value of eye 170 decreases (e.g., the focal length of eye 170 and/or the eye lens increases), the position of lens 138 is changed (e.g., lens 138 moves away from display 136) without changing the power of lens 138, the power of lens 138 is changed (e.g., the power of lens 138 increases) without moving the position of lens 138, or the position of lens 138 is changed and the power of lens 138 is changed (e.g., lens 138 moves away from display 136 and the power of lens 138 increases, lens 138 moves toward display 136 and the power of lens 138 increases, or lens 138 moves away from display 136 and the power of lens 138 decreases). In FIG. 9E, the power of lens 138 is increased and lens 138 is moved away from display device 136.

Thus, when display device 136 displays certain information (e.g., time of a day, such as “4:45 PM”), the displayed information will remain in focus on the retina of eye 170 (based on the adjustment of the display device based on the refraction value of eye 170). For example, the display device (and/or the associated optical elements) is adjusted so that the time of a day remains in focus regardless of whether the eye is focusing on a near object (e.g., having a high refraction value) or a far object (e.g., having a low refraction value). In some embodiments, a position of the certain information displayed on display device 136 is adjusted to maintain the certain information at a same location when perceived by eye 170 (e.g., lateral movement of the certain information caused by the changes to the optics, such as the changes to the position of lens 138 and/or the power of lens 138, is compensated by the adjustment of the position of the certain information on display device 136). In some embodiments, a size of the certain information displayed on display device 136 is adjusted to maintain the size of the certain information when perceived by eye 170 (e.g., changes to the size of the certain information caused by the changes to the optics, such as the changes to the position of lens 138 and/or the power of lens 138, is compensated by the adjustment of the size of the certain information on display device 136). This can be used in an augmented reality device or a virtual reality device to present the certain information in focus regardless of the refraction value of eye 170.

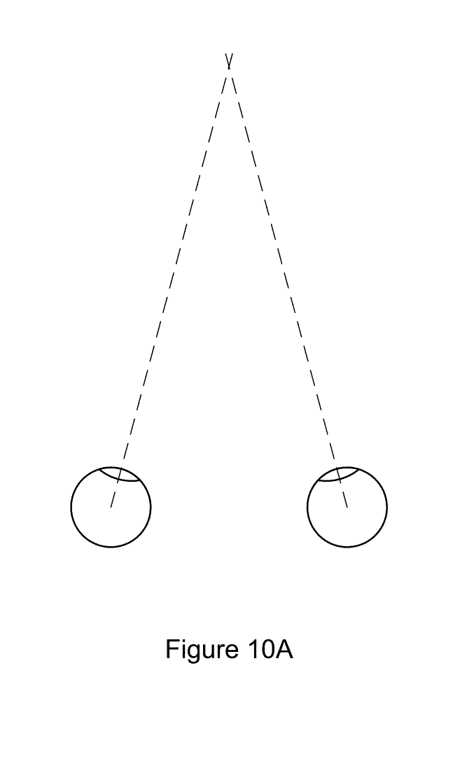

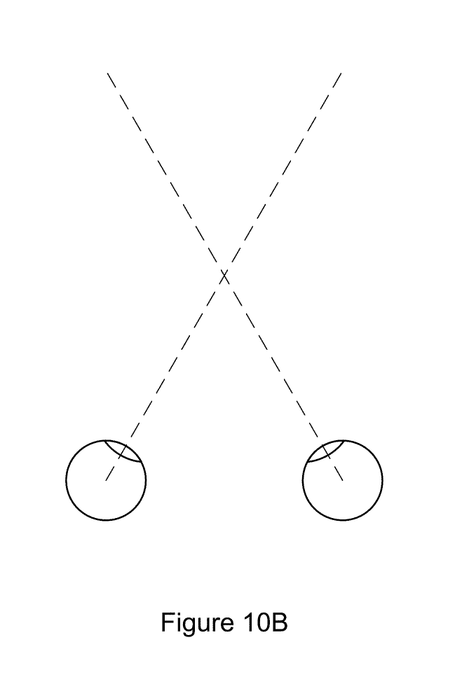

FIGS. 10A-10B illustrate a vergence of the eyes in accordance with some embodiments.

When the eyes focus on an object, the eyes have a particular vergence (e.g., 2 degrees), as shown in FIG. 10A. When the eyes focus on an object that is located closer, the eyes have a different vergence (e.g., 4 degrees). In some embodiments, the vergence of the eyes is determined by one or more pupil cameras. For example, a pupil camera collects an image of the an eye (e.g., a pupil of the eye), and based on the rotational movement of the eye (and/or the lateral movement of the pupil), the vergence is determined based on the rotational movement of the eye (and/or the lateral movement of the pupil). In some embodiments, an image of a left eye and an image of a right eye are used collectively to determine the vergence of the eyes (e.g., differentiate the rotational movement of the eyes for tracking).

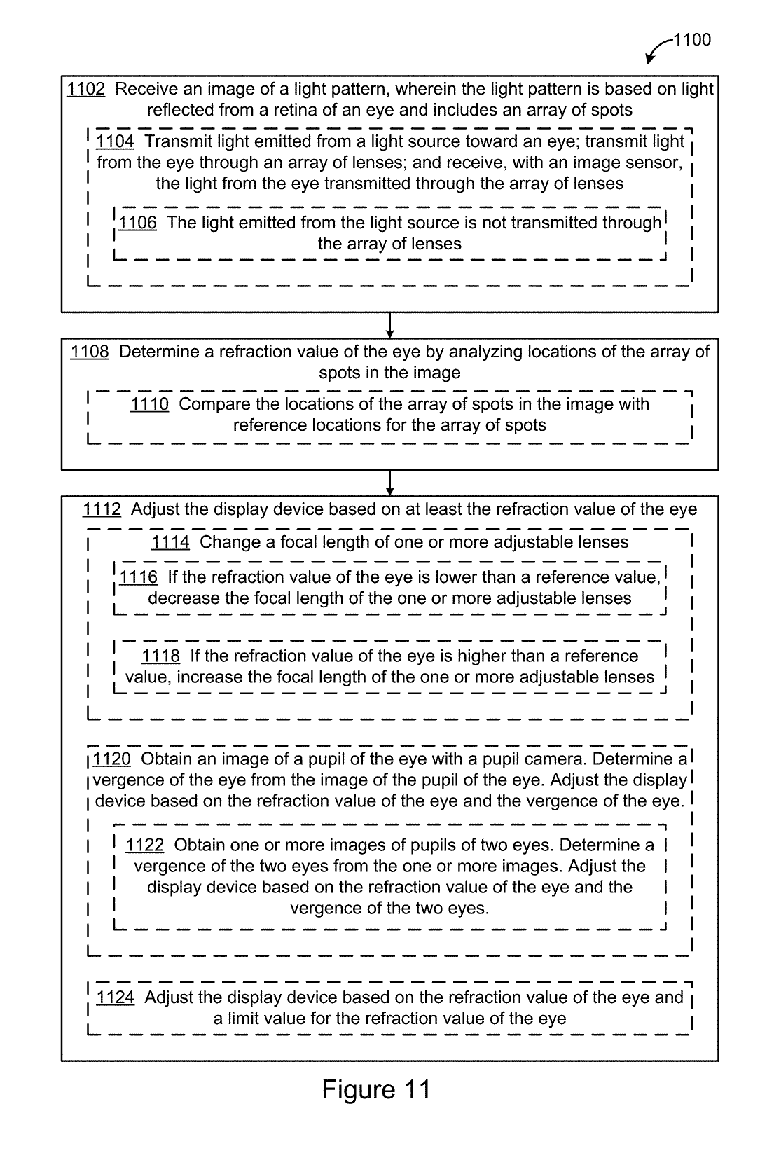

FIG. 11 is a flowchart representing method 1100 of determining a refraction value of an eye and adjusting a display device in accordance with some embodiments.

Method 1100 is performed at an electronic device (e.g., device 100 or device 102) with one or more processors, memory, and a display device.

The method includes (1102) receiving an image of a light pattern (e.g., an array of spots as shown in FIGS. 8A-8D). The light pattern is based on light reflected from a retina of an eye and includes an array of spots. In some embodiments, the light pattern does not include an array of spots (e.g., the light pattern includes multiple rings, such as multiple concentric rings).

In some embodiments, receiving the image of the light pattern includes (1104): transmitting light emitted from a light source (e.g., light source 120 in FIG. 1I) toward an eye; transmitting light from the eye through an array of lenses (e.g., an array of lenses 132 in FIG. 1J); and receiving, with an image sensor (e.g., image sensor 140 in FIG. 1J), the light from the eye transmitted through the array of lenses.

In some embodiments, the light emitted from the light source is not transmitted (1106) through the array of lenses (e.g., the light emitted from light source 120 in FIG. 1I is not transmitted through the array of lenses 132 before reaching eye 170 or before exiting from device 102).

The method also includes (1108) determining a refraction value of the eye by analyzing locations of the array of spots in the image (e.g., a spacing between the spots is calculated and the refraction value is determined based on the spacing between the spots, as illustrated in FIGS. 8A-8D).

In some embodiments, determining the refraction value of the eye includes (1110) comparing the locations of the array of spots in the image with reference locations for the array of spots (e.g., a spacing of the array of spots is compared with a reference spacing). In some embodiments, a location of a respective spot is compared with a reference location of a corresponding spot, and a displacement of the respective spot is calculated to determine the refraction value (e.g., an average displacement is used to determine the refraction value).

The method further includes (1112) adjusting the display device based on at least the refraction value of the eye (e.g., as illustrated in FIGS. 9A-9E, the display device is adjusted based on the refraction value of the eye).

In some embodiments, adjusting the display device includes adjusting one or more optical components associated with the display device.

In some embodiments, the display device includes an array of light emission elements (e.g., an array of liquid crystal display pixels) and one or more adjustable lenses.

In some embodiments, adjusting the display device includes (1114) changing a focal length of the one or more adjustable lenses.

In some embodiments, the method includes, in accordance with a determination that the refraction value of the eye is lower than a reference value, (1116) decreasing the focal length of the one or more adjustable lenses (e.g., FIGS. 9D-9E).

In some embodiments, the method includes, in accordance with a determination that the refraction value of the eye is higher than a reference value, (1118) increasing the focal length of the one or more adjustable lenses (e.g., FIGS. 9B-9C).

In some embodiments, adjusting the display device includes moving at least one lens of the one or more adjustable lenses (e.g., FIGS. 9B-9E).

In some embodiments, adjusting the display device includes displaying, on the display device, content that is selected based on the refraction value of the eye (e.g., the size of the content is selected based on the refraction value of the eye and/or the position of the content is selected based on the refraction value of the eye).

In some embodiments, the electronic device is coupled with a pupil camera. The method includes: (1120) obtaining an image of a pupil of the eye with the pupil camera; determining a vergence of the eye from the image of the pupil of the eye (e.g., based on the rotational movement and/or the lateral displacement of the eye or the pupil); and adjusting the display device based on at least the refraction value of the eye and the vergence of the eye. Having simultaneous measurements of the accommodation and the vergence of the eye allows adjustment of the display device based on the accommodation and the vergence of the eye, thereby preventing and/or reducing motion-sickness-like symptoms caused by a mismatch in the accommodation and the vergence response of the eye, which leads to neurological strain and asthenopia.

In some embodiments, the method includes: (1122) obtaining one or more images of pupils of two eyes; determining a vergence of the two eyes from the one or more images (which reduces the impact of the eye movement for tracking); and adjusting the display device based on at least the refraction value of the eye and the vergence of the two eyes.

In some embodiments, adjusting the display device based on at least the refraction value of the eye includes (1124) adjusting the display device based on the refraction value of the eye and a limit value for the refraction value of the eye. For example, the amplitude (e.g., power) of accommodation is measured prior to the use of the portable device by establishing a target at different distances and measuring the refraction value of an eye focusing on the target at different distances. Some users may not be able to focus on a target at a particular distance (e.g., due to myopia and/or hyperopia), and thus, a limit value is obtained (e.g., a particular user's eye cannot have a refraction value above +5 diopters). When adjusting the display device based on the refraction value, the display device will not be adjusted to require a refraction value of the eye that exceeds the limit value. This will increase the ocular comfort of the user.

The foregoing description, for purpose of explanation, has been described with reference to specific embodiments. However, the illustrative discussions above are not intended to be exhaustive or to limit the scope of claims to the precise forms disclosed. Many modifications and variations are possible in view of the above teachings. The embodiments were chosen and described in order to best explain the principles of the various described embodiments and their practical applications, to thereby enable others skilled in the art to best utilize the invention and the various described embodiments with various modifications as are suited to the particular use contemplated.

What is claimed is:

1. A method, comprising:
at an electronic device with one or more processors, memory, and a display device coupled with the one or more processors:
receiving an image of a light pattern, wherein the light pattern is based on light reflected from a retina of an eye and includes an array of spots;
determining a refraction value of the eye by analyzing locations of the array of spots in the image; and
adjusting the display device based on at least the refraction value of the eye.

2. The method of claim 1, wherein:
receiving the image of the light pattern includes:
transmitting light emitted from a light source toward an eye;
transmitting light from the eye through an array of lenses; and
receiving, with an image sensor, the light from the eye transmitted through the array of lenses.

3. The method of claim 2, wherein:
the light emitted from the light source is not transmitted through the array of lenses.

4. The method of claim 1, wherein:
determining the refraction value of the eye includes comparing the locations of the array of spots in the image with reference locations for the array of spots.

5. The method of claim 1, wherein:
the display device includes an array of light emission elements and one or more adjustable lenses; and
adjusting the display device includes changing a focal length of the one or more adjustable lenses.

6. The method of claim 5, including:
in accordance with a determination that the refraction value of the eye is lower than a reference value, decreasing the focal length of the one or more adjustable lenses.

7. The method of claim 5, including:
in accordance with a determination that the refraction value of the eye is higher than a reference value, increasing the focal length of the one or more adjustable lenses.

8. The method of claim 1, wherein:
the electronic device is coupled with a pupil camera; and
the method includes:
obtaining an image of a pupil of the eye with the pupil camera;
determining a vergence of the eye from the image of the pupil of the eye; and
adjusting the display device based on at least the refraction value of the eye and the vergence of the eye.

9. The method of claim 8, including:
obtaining one or more images of pupils of two eyes;
determining a vergence of the two eyes from the one or more images; and
adjusting the display device based on at least the refraction value of the eye and the vergence of the two eyes.

10. The method of claim 1, wherein:
adjusting the display device based on at least the refraction value of the eye includes adjusting the display device based on the refraction value of the eye and a limit value for the refraction value of the eye.

11. An electronic device, comprising:
one or more processors; and
memory storing one or more programs for execution by the one or more processors, the one or more programs including instructions for:
receiving an image of a light pattern, wherein the light pattern is based on light reflected from a retina of an eye and includes an array of spots;
determining a refraction value of the eye by analyzing locations of the array of spots in the image; and
adjusting the display device based on at least the refraction value of the eye.

12. The device of claim 11, wherein:
receiving the image of the light pattern includes:
transmitting light emitted from a light source toward an eye;
transmitting light from the eye through an array of lenses; and
receiving, with an image sensor, the light from the eye transmitted through the array of lenses.

13. The device of claim 11, wherein:
determining the refraction value of the eye includes comparing the locations of the array of spots in the image with reference locations for the array of spots.

14. The device of claim 11, wherein:
the display device includes an array of light emission elements and one or more adjustable lenses; and
adjusting the display device includes changing a focal length of the one or more adjustable lenses.

15. The device of claim 11, wherein:
the electronic device is coupled with a pupil camera; and
the one or more programs include instructions for:
obtaining an image of a pupil of the eye with the pupil camera;
determining a vergence of the eye from the image of the pupil of the eye; and
adjusting the display device based on at least the refraction value of the eye and the vergence of the eye.

16. A computer readable storage medium storing one or more programs for execution by one or more processors of an electronic device, the one or more programs including instructions for:
receiving an image of a light pattern, wherein the light pattern is based on light reflected from a retina of an eye and includes an array of spots;
determining a refraction value of the eye by analyzing locations of the array of spots in the image; and
adjusting the display device based on at least the refraction value of the eye.

17. The computer readable storage medium of claim 16, wherein:
receiving the image of the light pattern includes:
transmitting light emitted from a light source toward an eye;
transmitting light from the eye through an array of lenses; and
receiving, with an image sensor, the light from the eye transmitted through the array of lenses.

18. The computer readable storage medium of claim 16, wherein:
determining the refraction value of the eye includes comparing the locations of the array of spots in the image with reference locations for the array of spots.

19. The computer readable storage medium of claim 16, wherein:
adjusting the display device includes changing a focal length of one or more adjustable lenses of the display device.

20. The computer readable storage medium of claim 16, wherein the one or more programs include instructions for:
initiating a pupil camera to obtain an image of a pupil of the eye;
determining a vergence of the eye from the image of the pupil of the eye; and
adjusting the display device based on at least the refraction value of the eye and the vergence of the eye.
